(19) United States
(12) Patent Application Publication
(10) Pub. No.: US 2026/0077202 A1
(43) Pub. Date: Mar. 19, 2026

(54) SYSTEMS AND METHODS FOR ADMINISTERING AND ASSESSING BRAIN STIMULATION

(71) Applicant: Duke University, Durham, NC (US)

(72) Inventors: Warren Grill, Durham, NC (US); Kay Palopoli, Durham, NC (US)

(21) Appl. No.: 19/128,603

(22) PCT Filed: Nov. 10, 2023
(86) PCT No.: PCT/US2023/079353

§ 371 Date: May 9, 2025

Related U.S. Application Data
(60) Provisional application No. 63/383,354, filed on Nov. 11, 2022.

(51) Int. Cl. A61N 1/36; A61N 1/372

(52) U.S. Cl. CPC A61N 1/36139; A61N 1/36067; A61N 1/37241

(57) ABSTRACT

Embodiments of present disclosure provide systems and methods relating to neuromodulation. In particular, the present disclosure provides systems and methods for administering deep brain stimulation (DBS), and for evaluating the efficacy and efficiency of DBS for the treatment of neurological conditions (e.g., Parkinson's disease).

### RELATED APPLICATIONS

This application claims priority to and the benefit of U.S. Provisional Patent Application No. 63/383,354 filed Nov. 11, 2022, which is incorporated herein by reference in its entirety for all purposes.

### GOVERNMENT FUNDING

This invention was made with Government support under Federal Grant No. R37 NS040894 awarded by the National Institutes of Health (NIH). The Federal Government has certain rights to the invention.

### FIELD

The present disclosure provides systems and methods relating to neuromodulation. In particular, the present disclosure provides systems and methods for administering deep brain stimulation (DBS), and for evaluating the efficacy and efficiency of DBS for the treatment of neurological conditions (e.g., Parkinson's disease).

### BACKGROUND

Nervous Deep brain stimulation (DBS) is an effective surgical therapy for movement disorders such as Parkinson's Disease (PD) in individuals for whom pharmacological intervention alone either no longer manages their motor symptoms effectively or causes unwanted side effects. The current clinical standard is to deliver open-loop, constant rate DBS at frequencies above 100 Hz (typically 130-180 Hz) with a pre-set amplitude and pulse duration. Although open-loop DBS is very effective in treating the motor symptoms of PD, closed-loop DBS may maximize therapeutic benefit together with reducing side effects and energy use. In closed-loop DBS, symptom biomarkers can be implemented as control variables for algorithms to adjust stimulation parameters in real time. Closed-loop DBS thus has the potential to target a patient's dominant symptom, which may change dynamically throughout the day, fluctuating with the patient's state of activity. For symptom biomarkers to be successful feedback control signals for closed-loop DBS, they need to reflect reliably symptom severity on an appropriate time scale.

Neural biomarkers (recorded from either the basal ganglia or the cortex during DBS) are of particular interest because, with appropriate integrated recording circuitry, they do not require external hardware to measure motor symptoms (such as an accelerometer to capture tremor). Spontaneous oscillatory activity occurring in the local field potential (LFP) and DBS local evoked potentials (DLEPs) are both promising neural biomarkers for closed-loop DBS in PD. For example, oscillatory activity in the beta (13-35 Hz) and theta (4-7 Hz) bands are correlated with bradykinesia and resting tremor, respectively, and activity in the gamma band (60-90 Hz) correlates with dyskinesia (a common side effect during DBS). Similarly, DLEPs change dynamically in response to effective DBS with a time course that parallels changes in symptoms, and thus have the potential to serve as symptom biomarkers. However, clinically effective DBS frequencies provide a very short window (e.g., 5.4 ms at 185 Hz) for acquisition of the biomarker signal due to short inter-pulse intervals and a typical large artifact following each stimulation pulse.

### SUMMARY

Embodiments of the present disclosure include a method of administering brain stimulation therapy to a subject. In accordance with these embodiments, the method includes delivering a temporally non-regular pattern of electrical stimulation to the subject's brain; measuring at least one neural biomarker in the local field potential (LFP); and treating at least one symptom in the subject.

In some embodiments, administering the brain stimulation to the subject comprises administering deep brain stimulation (DBS), epidural cortical stimulation, epicortical cortical stimulation, transcranial electrical stimulation, and/or transcranial magnetic stimulation.

In some embodiments, administering the brain stimulation to the subject comprises administering closed-loop brain stimulation.

In some embodiments, the at least one neural biomarker comprises deep brain stimulation local evoked potential (DLEP) and/or oscillatory activity.

In some embodiments, measuring the at least one neural biomarker comprises recording single-neuron activity.

In some embodiments, the subject has or is suspected of having a neurological disease or disorder. In some embodiments, the neurological disease or disorder comprises dystonia, epilepsy, essential tremor, Parkinson's disease, Tourette syndrome, obsessive-compulsive disorder, depression, stroke, chorea, chronic pain, cluster headache, dementia, addiction, tinnitus, and/or obesity.

In some embodiments, the temporally non-regular pattern of electrical stimulation comprises at least one time period in which electrical stimulation is absent. In some embodiments, the at least one time period in which electrical stimulation is absent is from about 20 ms to about 200 ms in length. In some embodiments, the at least one time period in which electrical stimulation is absent occurs about every 100 ms to about every 5000 ms.

In some embodiments, the at least one neural biomarker comprises the DLEP, and wherein the DLEP is measured during the at least one time period in which electrical stimulation is absent.

In some embodiments, the at least one neural biomarker comprises DLEP, and wherein measuring the DLEP comprises measuring at least one of amplitude, latency, cycle number, and/or frequency.

In some embodiments, the at least one neural biomarker comprises oscillatory activity, and wherein oscillatory activity is measured during the at least one time period in which electrical stimulation is absent. In some embodiments, measuring oscillatory activity comprises measuring at least one of alpha, beta, gamma, and/or theta oscillations.

In some embodiments, treating at least one symptom in the subject comprises suppressing one or more of bradykinesia, tremor, and/or rigidity. In some embodiments, treating at least one symptom in the subject comprises suppressing at least one of alpha, beta, gamma, and/or theta oscillations.

In some embodiments, the method further comprises adjusting one or more stimulation parameters based on information obtained from measuring at least one neural biomarker. In some embodiments, the at least one neural biomarker comprises DLEP and/or oscillatory activity, and wherein the information obtained from measuring the DLEP and/or oscillatory activity indicates a need to adjust one or more stimulation parameters to treat at least one symptom in the subject. In some embodiments, the one or more stimulation parameters comprises electrode placement, electrode contact selection, stimulation parameter selection, and/or closed-loop control.

In some embodiments, the at least one neural biomarker comprises DLEP and/or oscillatory activity, and wherein the information obtained from measuring the DLEP and/or oscillatory activity indicates where to place an electrode in a subject's brain. In some embodiments, the at least one neural biomarker comprises DLEP and/or oscillatory activity, and wherein the information obtained from measuring the DLEP and/or oscillatory activity facilitates optimizing electrode configuration in a subject's brain. In some embodiments, determining electrode placement and/or optimizing electrode configuration comprises optimizing treatment for at least one symptom in the subject.

Embodiments of the present disclosure also include a system for administering brain stimulation therapy to a subject. In accordance with these embodiments, the system includes at least one implantable electrode configured to deliver electrical stimulation and to record electrical signals in the subject's brain; and a pulse generator electronically coupled to the at last one implantable electrode, wherein the pulse generator is configured to deliver a temporally non-regular pattern of electrical stimulation and measure at least one neural biomarker in the local field potential (LFP).

In some embodiments, the system administers at least one of deep brain stimulation (DBS), epidural cortical stimulation, epicortical cortical stimulation, transcranial electrical stimulation, and/or transcranial magnetic stimulation to the subject.

In some embodiments, the system administers closed-loop brain stimulation to the subject.

In some embodiments of the system, the at least one neural biomarker comprises deep brain stimulation local evoked potential (DLEP) and/or oscillatory activity.

In some embodiments, the system is configured to measure single-neuron activity.

In some embodiments of the system, the temporally non-regular pattern of electrical stimulation comprises at least one time period in which electrical stimulation is absent. In some embodiments of the system, the at least one neural biomarker comprises DLEP, and wherein DLEP is measured during the at least one time period in which electrical stimulation is absent. In some embodiments of the system, the at least one neural biomarker comprises oscillatory activity, and wherein oscillatory activity is measured during the at least one time period in which electrical stimulation is absent.

### BRIEF DESCRIPTION OF THE DRAWINGS

FIGS. 1A-1E: Methods to measure the effects of temporal patterns of deep brain stimulation on the motor symptoms of Parkinson's disease. A) Four stimulation patterns were tested: two fixed-frequency and two temporally non-regular patterns. IPF: instantaneous pulse frequency. B) Intraoperative timeline for assessment of tremor in tremor-dominant cohort. C) Accelerometer captured changes in tremor during DBS Off and On. D) Intraoperative timeline for assessment of alternating finger clicking of a two-button mouse in bradykinesia-dominant cohort. E) Clicking task captured changes in bradykinesia during DBS Off and On. The purple and green bars indicate time periods during which the middle or index fingers were pressing down on the mouse buttons, respectively. The black bars indicate time periods during which both fingers were pressing the mouse buttons.

FIGS. 2A-2D: Methods to measure the effects of temporal patterns of STN deep brain stimulation on local field potentials (LFPs) and DBS local evoked potentials (DLEPs). A) Signal processing pipeline of recorded brain signals. LFP activity was continuously analyzed during Absence whereas the DLEPs were only monitored during stimulation gaps. B) Quantification of DLEPs. C) Template subtraction for quantification of oscillatory activity. D) Spectrograms during DBS Off and On reveal modulation of both low (13-20 Hz) and high (20-35 Hz) beta sub-bands during DBS.

FIG. 3: Effects of different temporal patterns of STN DBS on tremor in tremor-dominant cohort. Each color is a different participant. Open circles represent data that were not included in the statistical analysis. Global ANOVA detected a significant interaction between stimulation pattern and measure (theta power and tremor, p<0.02), prompting us to subdivide the data between theta power and tremor. Fisher's Protected LSD post-hoc tests on tremor (shown here) distinguished High from Low (p<0.009) and Off (p<0.02), and Absence from Low (p<0.01) and Off (p<0.02). Student's t post-hoc tests on theta power (FIG. 8) found no effect of stimulation on the LFP. See below for complete statistical report. * p<0.05. ** p<0.01. N.S. Not significant.

FIGS. 4A-4B: Effects of different temporal patterns of STN DBS on beta oscillatory power (A) and bradykinesia (B) in bradykinesia-dominant cohort. Each color is a different participant. Open circles represent data that were not included in the statistical analysis. Global ANOVA detected a main effect of stimulation pattern (p<6×10⁻⁵), but no interaction between stimulation pattern and measure (beta power and bradykinesia). Tukey HSD post-hoc tests distinguished Off from Absence (p<2×10⁻⁴), Presence (p<4×10⁻), and High (p<6×10⁻⁴). The data were not further subdivided to protect against multiple comparisons. See below for complete statistical report. *** p<0.001.

FIGS. 5A-5C: Correlation between bradykinesia as assessed by alternating finger clicking and beta oscillatory activity in the STN during Off, High, Absence, and Presence. Each color is a different participant. A) Raw correlation between beta power and bradykinesia. B) Principal component analysis (PCA) identified interpatient variability. Loadings are plotted for each variable. C) Adjusted data revealed positive correlation between beta power and bradykinesia.

FIGS. 6A-6E: Characteristics of DBS local evoked potentials (DLEPs) in the STN evoked by different temporal patterns of STN DBS. A) Long-duration DLEPs were modulated (decline in amplitude and increase in latency) over time during Absence, but not Low. B) The number of DLEP periods present in the first 2 seconds after onset of Absence were positively correlated with the signal-to-noise (SNR) ratio of the recording. DLEPs recorded during IPG replacements had a lower SNR than those recorded during DBS implantations. C,D,E) Each set of points connected by a line is a different participant. Open circles represent data that were not included in the statistical analysis. C,D) Global ANOVA detected a significant (p<0.05) interaction term between time, stimulation pattern, and measure (number of periods and P1 amplitude). The data were therefore subdivided between number of periods (C) and P1 amplitude (D). C) ANOVA revealed a significant interaction between time and stimulation pattern (p<0.02). Paired t tests revealed that the number of DLEP periods decreased between 1 and 51 s for Absence (p<4×10⁻⁴) but not for Low, and that Absence produced more DLEP periods compared to Low at 1 s (p<0.04) but not at 51 s. D) ANOVA revealed a significant interaction between time and stimulation pattern (p<4×10−4). Paired t tests revealed that P1 amplitude decreased between 1 and 51 s for Absence (p<2×10⁻⁴) but not for Low. E) Global ANOVA on P1 latency detected a significant interaction between time and stimulation pattern (p<0.003). Subdividing the data by stimulation pattern, paired t tests revealed that P1 latency increased between 1 and 51 s since stimulation onset for High (p<4×10⁻⁴), Absence (p<0.002), and Presence (p<2×10⁻⁴), but not for Low. Subdividing the data by time, a 1-way ANOVA detected an effect of stimulation on P1 latency at 1 s (p<0.02) but not 51 s. Tukey HSD post-hoc tests on P1 latency at 1 s distinguished Low from High (p<0.02), Absence (p<0.05), and Presence (p<0.03). See below for complete statistical report. * p<0.05. ** p<0.01. *** p<0.001. N.S. Not significant.

FIG. 7: Baseline tremor does not worsen during subsequent Off trials. Each line is a different participant. Open circles represent data that were not included in the statistical analysis. Global ANOVA found no effect of trial number on tremor. Since there was no worsening of tremor over time during Off trials, data from multiple experimental blocks (up to 3) were averaged. See below for complete statistical report.

FIG. 8: Theta (top), alpha (middle), and beta (bottom) power were not modulated during any stimulation pattern in the tremor-dominant cohort. Global ANOVA did not reveal either a main effect of stimulation pattern on the LFP or an interaction between stimulation pattern and LFP frequency. See below for complete statistical report.

FIG. 9: Baseline bradykinesia does not worsen during subsequent Off trials for either the index or middle finger. Each line is a different participant. Open circles represent data that were not included in the statistical analysis. Global ANOVA did not reveal either a main effect of trial number on bradykinesia or an interaction between trial number and finger. Since there was no worsening of bradykinesia over time during Off trials, data from multiple Off trials were averaged. See below for complete statistical report.

FIG. 10: An effect of time since stimulation onset on bradykinesia was not detected for either the index or middle finger. Each line is a different participant. Open circles represent data that were not included in the statistical analysis. 3-way global ANOVA (factors: finger, epoch, stimulation pattern) did not reveal any main effects or interactions. Since an effect of epoch number on bradykinesia was not detected, click durations during all three epochs of a trial were pooled before CV calculation. See below for complete statistical report.

FIG. 11: Bradykinesia was quantified for the middle and index finger separately during Off trials (each point represents one participant). The black unity line represents the boundary where both fingers performed equally. Blue circles represent participants for whom the middle finger performed worst; red circles represent participants for whom the index finger performed worst. The worst-performing finger for each participant was selected for all subsequent analysis.

FIG. 12: DLEPs are modulated over time during High, Absence, and Presence, but not Low. These data show the first 5 ms post stimulus (averaged in 2 s, non-overlapping windows and filtered) in one individual across each stimulation pattern. P1 declines in amplitude and increases in latency between 5 and 55 s, but not 55 and 295 s, for High, Absence, and Presence. During Low, P1 amplitude and latency remains constant between all three time points.

FIG. 13: Bath testing of recording instrumentation during Absence stimulation. A function generator was programmed to deliver mock evoked activity (4 cycles of a 1 kHz sine wave) after every stimulation pulse. The amplifier was able to detect the mock evoked activity (<5 ms), and past 5 ms, the amplifier did not introduce or pick up any artifacts. Therefore, long-latency (>5 ms) DLEPs during Absence stimulation gaps are not a ringing artifact from the instrumentation.

FIG. 14: DLEPs of 50 ms duration during clinically effective DBS (Absence). The top panel shows the time evolution of the DLEPs over the course of an entire trial (300 s). These data were processed as illustrated in FIG. 2B (averaged in 2 s, non-overlapping windows, and filtered). The bottom two panels zoom in on the first 5 and 1 s of the trial, respectively, and show an overlay of the raw data (not averaged). The raw data in the first second of the trial reveal DLEPs of 50 ms duration.

### DETAILED DESCRIPTION

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse), a non-human primate (e.g., a monkey, such as a cynomolgus or rhesus monkey, chimpanzee, etc.) and a human. In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Therapy” and/or “therapy regimen” generally refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

“Evoked potential” or “EP” as used herein generally refers to the electrical potential in a specific pattern recorded from a specific part of the nervous system of a subject. EPs measure the electrophysiologic responses of the nervous system of a subject to various stimuli. EPs can be used as a means for assessing the effects of stimulation parameter settings by electrically recording the aggregate neural activated evoked following stimulation. EPs are also referred to as evoked compound action potential (ECAP), or DBS local evoked potential (DLEP). For the purposes of the present disclosure, the term “evoked potential” refers to any of these types of recordings. As described further herein, various characteristics of an EP can be used as a means for selecting or adjusting one or more stimulation parameter settings.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. BRAIN STIMULATION METHODS

Temporally non-regular patterns of stimulation allowed for the monitoring, for the first time, the entire duration of DLEPs (over 50 ms) during clinically effective DBS. This revealed a larger number of oscillatory periods (i.e., up to 18) as an additional DLEP feature that is modulated during DBS. Other investigations have suggested periodically omitting one pulse during fixed-frequency 130 Hz DBS to record several DLEP periods; however, this amounts to roughly 15 ms of DLEPs. In the present disclosure, up to 50 ms are monitored. The results of the present disclosure facilitate and expand the use of DLEPs as a tool for probing basal ganglia circuitry, as a signal to guide electrode placement and contact selection, as a signal to guide initial device programming, and as a feedback signal for closed-loop DBS.

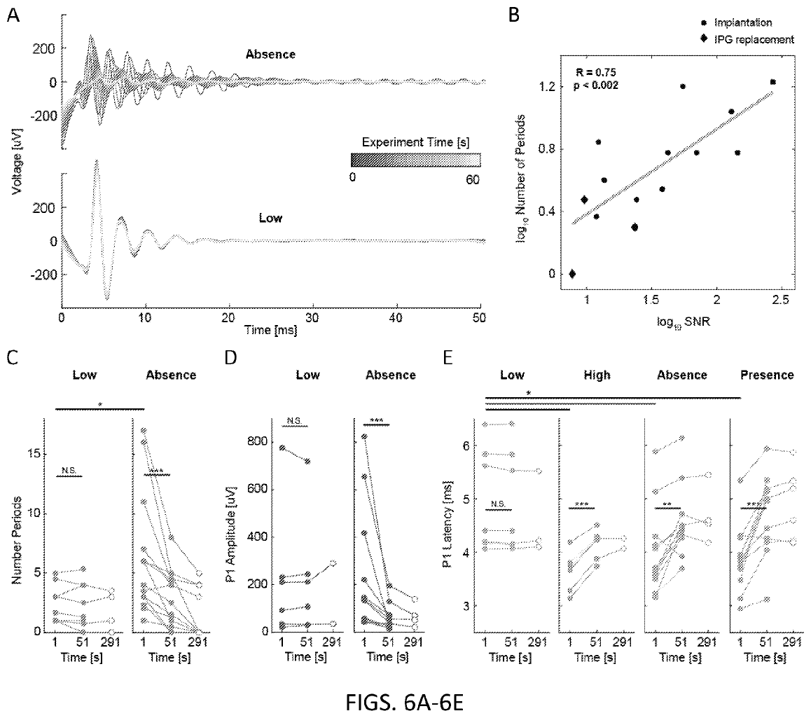

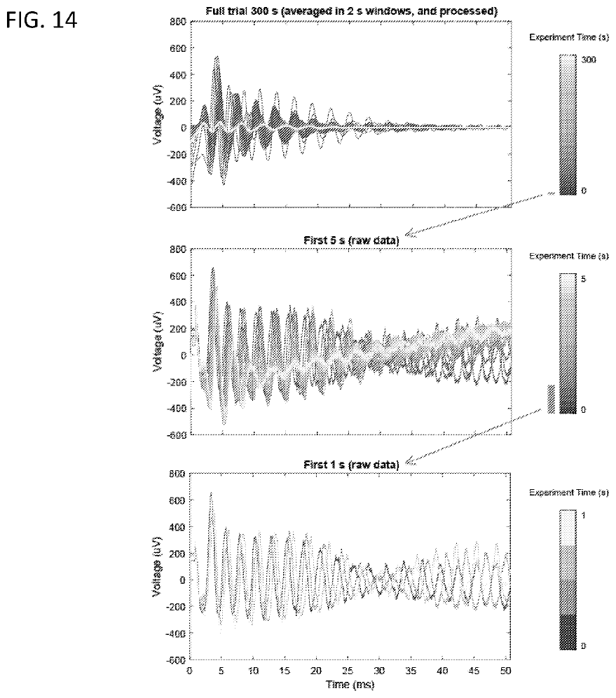

The repeating 50 ms gaps in Absence enable regular assessment of DLEPs more easily than traditional DBS. Clinically relevant DBS frequencies (>100 Hz) have short IPIs and thus only a limited number of DLEP periods (1-2) can be recorded continuously. Absence circumvents this limitation by providing a window of 50 ms to record the entirety of the DLEPs while maintaining effective symptom relief. DLEPs were recorded with 1-18 periods and with a duration as long as 50 ms during clinically effective DBS (FIG. 14). DLEPs are of particular interest as control variables for parameter programming and closed-loop applications of DBS because they have larger amplitudes (˜100s of μV, and up to 800 μV, FIG. 6D) compared to cortical evoked potentials (˜1-5 μV) and beta band activity (˜1-4 μV) and thus provide a higher SNR. Various features of DLEPs (such as amplitude, latency, and frequency) have garnered interest to inform electrode placement, electrode contact selection, stimulation parameter selection, and closed-loop control. Specifically, DLEP amplitude is greatest in the dorsal STN, which is the DBS target associated with the best symptom relief. DLEPs are also present under anesthesia, thus making them useful guides for electrode placement during surgery. Lastly, greater DLEP amplitudes are associated with superior symptom relief from DBS and therefore hold promise as tools for DBS parameter programming. However, most studies were unable to record DLEPs in their entirety; Absence creates a clear and compelling solution to this challenge.

The long-duration DLEPs recorded during Absence raise further questions about the effects of STN DBS on basal ganglia circuitry. First, Absence produces a greater number of periods compared to Low (FIGS. 6A and 6C). Second, the number of periods recorded during Absence is correlated with SNR (FIG. 6B), suggesting that long-duration DLEPs may be present in all patients receiving clinically effective DBS, albeit non-detectable due to low SNR. Previous work suggests that DLEPs arise from the reciprocal connections between the STN and the globus pallidus external (GPe) segment. The results of the present disclosure suggest that long-duration DLEPs may provide additional approaches for probing the basal ganglia circuitry: the greater the number of DLEP periods, the longer the reciprocal connections between STN and GPe are engaged. For example, for some participants, long-duration DLEPs entirely disappear before 291 s, while in others they do not (FIG. 6C). This discrepancy is not fully explained by interpatient differences in the number of DLEP periods recorded immediately after stimulation onset (which was determined to be correlated to SNR). Namely, the two participants with the highest number of initial DLEP periods (17 and 16, respectively) revealed different dynamics: by 291 s, the number of DLEP periods had decreased to 5 and 0, respectively (FIG. 6C). Therefore, the disappearance and time-to disappearance of long-durations DLEPs may be informative measures about basal ganglia circuitry dynamics during DBS and potentially provide a mechanistic explanation for observed clinical effects.

One aspect of the present disclosure is that while long-duration DLEPs may have utility for probing the basal ganglia circuitry, they appear to be more challenging to record reliably after leads have been implanted for years. These data highlight the detrimental effects of signal quality degradation years after DBS implantation when recording long-duration DLEPs. Participant 21 was enrolled in the study twice: during DBS implantation and during IPG replacement 5 years later. Even when recording DLEPs (the highest-amplitude neural biomarkers for DBS), the SNR of Participant 21 declined from 270 to 8 over the course of 5 years (however, it is important to note that although the original lead remained untouched during IPG replacement, different contacts were used for both stimulation and recording between the two data collection sessions). Signal quality deteriorates over time due to due to scar tissue formation surrounding the DBS lead as well as stimulation-induced changes at the tissue-electrode interface.

Both temporally non-regular patterns of DBS performed comparably to traditional fixed-frequency stimulation across all metrics evaluated (tremor, bradykinesia, oscillatory activity, and DLEPs). While the effects of Absence and Presence on the motor symptoms and LFP of persons with PD during STN DBS were not different from the effects of High, the results reinforce the promise of the temporal pattern of stimulation as an untapped parameter space. The same way programmable parameters of fixed-frequency DBS are tuned when an individual does not tolerate or respond to the stimulation, so can the temporal pattern be selected differentially according to patient response. Absence and Presence provide alternative settings that can tailor DBS to specific individuals and produced greater suppression of bradykinesia (in 4/11 and 5/10 participants, respectively) and tremor (in 3/7 and 2/8 participants, respectively) when compared to High.

Temporally non-regular patterns can also improve the efficiency of DBS by reducing the average stimulation frequency while maintaining clinical efficacy. However, the finding that clinical efficacy is maintained when introducing small gaps (˜50 ms) in the stimulation is specific to STN DBS for PD and does not generalize to other movement disorders and brain areas. Introducing gaps in stimulation significantly reduced the efficacy of DBS of the ventral intermediate (VIM) nucleus of the thalamus when treating essential tremor (ET). A computational model revealed that the stimulation gaps reduced efficacy by failing to mask burst-driver inputs to the thalamus. Conversely, these results show that when stimulating the STN, stimulation gaps of 50 ms do not reduce efficacy when treating PD (across both tremor and bradykinesia). This suggests that the impact of gaps in the stimulation when treating disease using DBS is dependent on the mechanism of action of DBS on the targeted neural circuit.

These data revealed a positive correlation between beta power and bradykinesia: the higher the beta power, the more severe the bradykinesia (R=0.54, p<4×10⁻⁴). Bradykinesia was quantified with a clicking task that is correlated to UPDRS motor scores. Since UPDRS is the current clinical standard for evaluating PD severity, the correlation found in the data was converted from clicking task performance to UPDRS motor scores. To predict the worsening of UPDRS motor score for a given increase in beta power, the slope of the correlation (0.19) was multiplied by the correlation coefficient (R=0.53) and a worsening of 0.1007 log ₁₀CV per order of magnitude increase in beta power was obtained. To convert the worsening of clicking task performance to a clinically interpretable scale, the established relationship between log ₁₀CV and the Unified Parkinson's Disease Rating Scale (UPDRS) III (motor scale) was used. The gain (80 UPDRS motor points per 1 log ₁₀CV unit) was multiplied by the correlation coefficient (R=0.66) to obtain the scaling factor (52.8 UPDRS motor points per 1 log ₁₀CV unit). This scaling factor was used to convert from log ₁₀CV to UPDRS motor points and reveal the following relationship: an increase of beta power by one order of magnitude results in a worsening of PD of approximately 5 UPDRS motor points. This analysis demonstrates that an increase in beta power has a clinically meaningful effect on the UPDRS motor scores of persons with PD.

These data confirm model predictions from previous work that Absence and Presence reduce beta activity compared to Off. However, the model also predicted that beta suppression was greater during Absence and Presence than High, and the intraoperative recordings are not consistent with this prediction. A potential explanation for this discrepancy is that the computational model did not explicitly include the striatum, the thalamus, and the cortex. While there have been many useful computational models of the basal ganglia circuitry, there is not a consensus as to which specific elements are essential for realistic models of STN DBS. However, human studies and patient-specific models suggest that the hyperdirect pathway from the cortex to the STN is implicated in symptom relief during STN DBS. Together, this might explain why the computational model that was used to evaluate Absence and Presence was accurate in predicting their effects compared to baseline (large difference in beta activity) but overestimated them compared to High (small difference in beta activity).

These data demonstrated that stimulation pattern had a statistically significant effect on P1 latency in the first second after stimulation onset (FIG. 6E). This finding is consistent with previous research showing that DLEP latency is variable across different paired pulse intervals. Differences in the instantaneous pulse frequency of the stimulation patterns (100 ms for Low, 5.4 ms for High, 5.1 ms for Absence, and 7.0 ms for Presence) are analogous to different paired pulse intervals.

One aspect of the present disclosure involves the challenges of proving a negative result (i.e., concluding that Absence and Presence ubiquitously have the same clinical performance as High). It is possible that Absence and Presence do have different effects than High, but the differences were too small to be detected in the data set, even after accounting for participant as a random factor in the statistical models. This is especially important to note for the tremor-dominant cohort, which contained fewer participants compared to the bradykinesia-dominant cohort (9 versus 12), making differences between groups harder to detect. Nonetheless, Absence and Presence did provide clinically relevant effects when compared to Low and Off. Therefore, the conclusions that temporally non-regular patterns can serve as an additional parameter space for preserving or increasing efficacy and efficiency, and provide a more reliable paradigm for biomarker quantification during closed-loop DBS, still stand.

The results of the present disclosure demonstrate the multi-faceted utility of temporally non-regular patterns of DBS. First, they serve as an additional parameter space that can increase the efficacy (degree of symptom relief) of DBS on a patient-specific basis. Second, they can increase the efficiency (energy required for delivery of therapy) of DBS by reducing the average stimulation frequency. Lastly, they enable us to monitor DLEPs throughout their entire duration while maintaining motor symptom management by DBS, thus providing an improved paradigm for biomarker quantification during closed-loop DBS. These results also reveal new temporal dynamics of DLEPs in response to clinically effective DBS, which can serve as an additional tool for probing the basal ganglia circuitry.

In accordance with the above, and as described further herein, embodiments of the present disclosure provide systems and methods relating to neuromodulation. In particular, embodiments of the present disclosure include a method of administering brain stimulation therapy to a subject. In some embodiments, the method includes delivering a temporally non-regular pattern of electrical stimulation to the subject's brain. In some embodiments, the method includes measuring at least one neural biomarker in the local field potential (LFP). In some embodiments, the method includes treating at least one symptom in the subject. As would be recognized by one of ordinary skill in the art based on the present disclosure, the method for administering brain stimulation therapy to a subject can include one or more of the aforementioned steps in any order or arrangement.

In some embodiments, administering brain stimulation to a subject includes administering deep brain stimulation (DBS) to the subject. In some embodiments, administering brain stimulation to a subject includes administering epidural cortical stimulation to the subject. In some embodiments, administering brain stimulation to a subject includes administering epicortical cortical stimulation to the subject. In some embodiments, administering brain stimulation to a subject includes administering transcranial electrical stimulation to the subject. In some embodiments, administering brain stimulation to a subject includes administering transcranial magnetic stimulation to the subject. In some embodiments, administering any of the aforementioned types of brain stimulation to a subject includes administering stimulation as part of stimulation protocol that provides feedback control during stimulation (e.g., closed-loop stimulation).

Evoked potentials may be a useful neural biomarker to aid in the ability to assess symptoms relating to a neurological disease or disorder, and also to assess treatment efficacy for those symptoms. As described further herein, deep brain stimulation local evoked potential (DLEP) can be used as one such neural biomarker comprises, and embodiments of the present disclosure include the ability to record the entire duration of a DLEP without compromising stimulation treatment. Additionally, brain (or neural) oscillations generally refer to the rhythmic and/or repetitive electrical activity generated spontaneously and in response to stimuli by neural tissue in the central nervous system. Therefore, brain oscillatory activity can also be used as a neural biomarker in accordance with the various embodiments of the present disclosure. DLEP and oscillatory activity can be used together or independently as neural biomarkers for assessing symptoms relating to a neurological disease or disorder, and also for assessing treatment efficacy and/or efficiency.

In some embodiments, the methods and systems of the present disclosure can be used to assess a subject that has or is suspected of having a neurological disease or disorder. In some embodiments, the neurological disease or disorder is dystonia, epilepsy, essential tremor, Parkinson's disease, Tourette syndrome, obsessive-compulsive disorder, depression, stroke, chorea, chronic pain, cluster headache, dementia, addiction, tinnitus, and/or obesity. In some embodiments, delivering a temporally non-regular pattern of electrical stimulation to the subject's brain treats one or more symptoms associated with one or more of the aforementioned neurological diseases or disorders. In some embodiments, measuring at least one neural biomarker (e.g., DLEP and/or oscillatory activity) in the local field potential (LFP) provides feedback to a clinician and regarding the efficacy (e.g., degree of symptom relief) of brain stimulation on a patient-specific basis, and/or the efficiency of the system (e.g., energy required for delivery of therapy). In some embodiments, treating at least one symptom in the subject comprises suppressing one or more of bradykinesia, tremor, and/or rigidity.

As described further herein, the temporally non-regular pattern of electrical stimulation administered to a subject can include at least one time period in which electrical stimulation is absent (e.g., a gap in electrical stimulation). In some embodiments, at least one such time period in which electrical stimulation is absent can be from about 20 ms to about 200 ms in length. In some embodiments, the time period in which electrical stimulation is absent is from about 50 ms to about 200 ms in length. In some embodiments, the time period in which electrical stimulation is absent is from about 75 ms to about 200 ms in length. In some embodiments, the time period in which electrical stimulation is absent is from about 100 ms to about 200 ms in length. In some embodiments, the time period in which electrical stimulation is absent is from about 125 ms to about 200 ms in length. In some embodiments, the time period in which electrical stimulation is absent is from about 150 ms to about 200 ms in length. In some embodiments, the time period in which electrical stimulation is absent is from about 175 ms to about 200 ms in length. In some embodiments, the time period in which electrical stimulation is absent is from about 50 ms to about 175 ms in length. In some embodiments, the time period in which electrical stimulation is absent is from about 50 ms to about 150 ms in length. In some embodiments, the time period in which electrical stimulation is absent is from about 50 ms to about 125 ms in length. In some embodiments, the time period in which electrical stimulation is absent is from about 50 ms to about 100 ms in length. In some embodiments, the time period in which electrical stimulation is absent is from about 50 ms to about 75 ms in length. In some embodiments, the time period in which electrical stimulation is absent is from about 75 ms to about 150 ms in length. In some embodiments, the time period in which electrical stimulation is absent is from about 100 ms to about 150 ms in length.

In some embodiments, the time period in which electrical stimulation is absent occurs about every 100 ms to about every 5000 ms. In some embodiments, the time period in which electrical stimulation is absent occurs about every 250 ms to about every 5000 ms. In some embodiments, the time period in which electrical stimulation is absent occurs about every 500 ms to about every 5000 ms. In some embodiments, the time period in which electrical stimulation is absent occurs about every 1000 ms to about every 5000 ms. In some embodiments, the time period in which electrical stimulation is absent occurs about every 1500 ms to about every 5000 ms. In some embodiments, the time period in which electrical stimulation is absent occurs about every 2000 ms to about every 5000 ms. In some embodiments, the time period in which electrical stimulation is absent occurs about every 100 ms to about every 2500 ms. In some embodiments, the time period in which electrical stimulation is absent occurs about every 3000 ms to about every 5000 ms. In some embodiments, the time period in which electrical stimulation is absent occurs about every 3500 ms to about every 5000 ms. In some embodiments, the time period in which electrical stimulation is absent occurs about every 4000 ms to about every 5000 ms. In some embodiments, the time period in which electrical stimulation is absent occurs about every 4500 ms to about every 5000 ms. In some embodiments, the time period in which electrical stimulation is absent occurs about every 100 ms to about every 4500 ms. In some embodiments, the time period in which electrical stimulation is absent occurs about every 100 ms to about every 4000 ms. In some embodiments, the time period in which electrical stimulation is absent occurs about every 100 ms to about every 3500 ms. In some embodiments, the time period in which electrical stimulation is absent occurs about every 100 ms to about every 3000 ms. In some embodiments, the time period in which electrical stimulation is absent occurs about every 100 ms to about every 2500 ms. In some embodiments, the time period in which electrical stimulation is absent occurs about every 100 ms to about every 2000 ms. In some embodiments, the time period in which electrical stimulation is absent occurs about every 100 ms to about every 1500 ms. In some embodiments, the time period in which electrical stimulation is absent occurs about every 100 ms to about every 500 ms. In some embodiments, the time period in which electrical stimulation is absent occurs about every 1000 ms to about every 4000 ms. In some embodiments, the time period in which electrical stimulation is absent occurs about every 2000 ms to about every 3500 ms. In some embodiments, the time period in which electrical stimulation is absent occurs about every 1500 ms to about every 3000 ms. In some embodiments, the time period in which electrical stimulation is absent occurs about every 3000 ms to about every 4500 ms.

In some embodiments, the at least one neural biomarker comprises the DLEP, and in accordance with these embodiments, the DLEP is measured during at least one time period in which electrical stimulation is absent (e.g., during a gap in electrical stimulation). In some embodiments, at least one neural biomarker comprises DLEP, and measuring the DLEP includes measuring at least one of amplitude, latency, cycle number, and/or frequency.

In some embodiments, the at least one neural biomarker comprises oscillatory activity, and in accordance with these embodiments, oscillatory activity is measured during at least one time period in which electrical stimulation is absent (e.g., during a gap in electrical stimulation). In some embodiments, measuring oscillatory activity includes measuring at least one of alpha, beta, gamma, and/or theta oscillations. In some embodiments, treating at least one symptom in the subject includes suppressing at least one of alpha, beta, gamma, and/or theta oscillations.

In some embodiments, the methods of the present disclosure include adjusting one or more stimulation parameters based on information obtained from measuring at least one neural biomarker. In some embodiments, at least one neural biomarker includes DLEP and/or oscillatory activity, and information obtained from measuring the DLEP and/or oscillatory activity indicates a need to adjust one or more stimulation parameters to treat at least one symptom in the subject. In some embodiments, the one or more stimulation parameters comprises electrode placement, electrode contact selection, stimulation parameter selection, and/or closed-loop control. In some embodiments, the one or more stimulation parameters includes stimulation pulse amplitude, stimulation pulse duration, stimulation pulse repetition rate, stimulation pulse shape, temporal pattern of stimulation pulse train, and stimulation duty cycle.

In some embodiments, at least one neural biomarker includes DLEP and/or oscillatory activity, and the information obtained from measuring the DLEP and/or oscillatory activity indicates to a clinician where to place an initial electrode in a subject's brain (e.g., during clinical evaluation). In some embodiments, at least one neural biomarker includes DLEP and/or oscillatory activity, and the information obtained from measuring the DLEP and/or oscillatory activity includes information as to where to place one or more electrodes (or adjust the position of one or more electrodes) to optimize treatment. In some embodiments, the information obtained from measuring the DLEP and/or oscillatory activity includes information for optimizing electrode configuration in a patient-specific manner. For example, optimizing electrode configuration can include, without limitation, adjusting one or more contacts on a multielectrode array that are active, and also adjusting amplitude and/or polarity. In some embodiments, determining the initial placement of an electrode and/or optimizing electrode configuration comprises enhances treatment (e.g., alleviates one or more symptoms in the subject).

### 3. BRAIN STIMULATION SYSTEMS

Embodiments of the present disclosure include a system for administering brain stimulation therapy to a subject. In accordance with these embodiments, the system includes at least one implantable electrode configured to deliver electrical stimulation and to record electrical signals in the subject's brain. The system also includes a pulse generator electronically coupled to at least one implantable electrode. In accordance with these embodiments, the pulse generator is configured to deliver a temporally non-regular pattern of electrical stimulation and measure at least one neural biomarker in the local field potential (LFP).

The systems described herein can be configured to administer any type of brain stimulation to a subject. In some embodiments, the system is configured to administer deep brain stimulation (DBS) to a subject. In some embodiments, the system is configured to administer epidural cortical stimulation to a subject. In some embodiments, the system is configured to administer epicortical cortical stimulation to a subject. In some embodiments, the system is configured to administer transcranial electrical stimulation to a subject. In some embodiments, the system is configured to administer transcranial magnetic stimulation to a subject. In some embodiments, the system is configured to administer any of the aforementioned types of brain stimulation to a subject as part of stimulation protocol that provides feedback control during stimulation (e.g., closed-loop stimulation). In some embodiments, the system is configured to measure single-neuron activity.

As would be recognized by one of ordinary skill in the art based on the present disclosure, the systems described herein are configured to both deliver electrical stimulation and to record electrical signals in a subject's brain, and therefore, are configured to account for various stimulation artefacts. For example, embodiments of the present disclosure can include instrumentation designed to suppress stimulus artefact, as described in Kent, et al., J Neural Eng. 2012 June; 9(3): 036004, which is incorporated herein by reference in its entirety and for all purposes.

In some embodiments, the systems of the present disclosure are configured to assess, measure, or quantify one or more neural biomarkers relating to a neurological disease or disorder. In some embodiments, the neural biomarker includes deep brain stimulation local evoked potential (DLEP) and/or oscillatory activity. DLEP and oscillatory activity can be used together or independently as neural biomarkers for assessing symptoms relating to a neurological disease or disorder, and also for assessing treatment efficacy and/or efficiency.

In accordance with these embodiments, the systems of the present disclosure are configured to deliver a temporally non-regular pattern of electrical stimulation to a subject. In some embodiments, the temporally non-regular pattern of electrical stimulation includes at least one time period in which electrical stimulation is absent. In some embodiments of the system, the neural biomarker being measured is DLEP, and the DLEP is measured during the time period in which electrical stimulation is absent. In some embodiments of the system, the neural biomarker being measured is brain oscillatory activity, and the oscillatory activity is measured during the time period in which electrical stimulation is absent.

As described further herein, the systems of the present disclosure are configured to deliver a temporally non-regular pattern of electrical stimulation to a subject comprising at least one time period in which electrical stimulation is absent (e.g., a gap in electrical stimulation). In some embodiments, at least one such time period in which electrical stimulation is absent can be from about 20 ms to about 200 ms in length. In some embodiments, the time period in which electrical stimulation is absent is from about 50 ms to about 200 ms in length. In some embodiments, the time period in which electrical stimulation is absent is from about 75 ms to about 200 ms in length. In some embodiments, the time period in which electrical stimulation is absent is from about 100 ms to about 200 ms in length. In some embodiments, the time period in which electrical stimulation is absent is from about 125 ms to about 200 ms in length. In some embodiments, the time period in which electrical stimulation is absent is from about 150 ms to about 200 ms in length. In some embodiments, the time period in which electrical stimulation is absent is from about 175 ms to about 200 ms in length. In some embodiments, the time period in which electrical stimulation is absent is from about 50 ms to about 175 ms in length. In some embodiments, the time period in which electrical stimulation is absent is from about 50 ms to about 150 ms in length. In some embodiments, the time period in which electrical stimulation is absent is from about 50 ms to about 125 ms in length. In some embodiments, the time period in which electrical stimulation is absent is from about 50 ms to about 100 ms in length. In some embodiments, the time period in which electrical stimulation is absent is from about 50 ms to about 75 ms in length. In some embodiments, the time period in which electrical stimulation is absent is from about 75 ms to about 150 ms in length. In some embodiments, the time period in which electrical stimulation is absent is from about 100 ms to about 150 ms in length.

In some embodiments, the systems of the present disclosure are configured to deliver a temporally non-regular pattern of electrical stimulation to a subject comprising at least one time period in which electrical stimulation is absent, and the time period in which electrical stimulation is absent occurs about every 100 ms to about every 5000 ms. In some embodiments, the time period in which electrical stimulation is absent occurs about every 250 ms to about every 5000 ms. In some embodiments, the time period in which electrical stimulation is absent occurs about every 500 ms to about every 5000 ms. In some embodiments, the time period in which electrical stimulation is absent occurs about every 1000 ms to about every 5000 ms. In some embodiments, the time period in which electrical stimulation is absent occurs about every 1500 ms to about every 5000 ms. In some embodiments, the time period in which electrical stimulation is absent occurs about every 2000 ms to about every 5000 ms. In some embodiments, the time period in which electrical stimulation is absent occurs about every 100 ms to about every 2500 ms. In some embodiments, the time period in which electrical stimulation is absent occurs about every 3000 ms to about every 5000 ms. In some embodiments, the time period in which electrical stimulation is absent occurs about every 3500 ms to about every 5000 ms. In some embodiments, the time period in which electrical stimulation is absent occurs about every 4000 ms to about every 5000 ms. In some embodiments, the time period in which electrical stimulation is absent occurs about every 4500 ms to about every 5000 ms. In some embodiments, the time period in which electrical stimulation is absent occurs about every 100 ms to about every 4500 ms. In some embodiments, the time period in which electrical stimulation is absent occurs about every 100 ms to about every 4000 ms. In some embodiments, the time period in which electrical stimulation is absent occurs about every 100 ms to about every 3500 ms. In some embodiments, the time period in which electrical stimulation is absent occurs about every 100 ms to about every 3000 ms. In some embodiments, the time period in which electrical stimulation is absent occurs about every 100 ms to about every 2500 ms. In some embodiments, the time period in which electrical stimulation is absent occurs about every 100 ms to about every 2000 ms. In some embodiments, the time period in which electrical stimulation is absent occurs about every 100 ms to about every 1500 ms. In some embodiments, the time period in which electrical stimulation is absent occurs about every 100 ms to about every 500 ms. In some embodiments, the time period in which electrical stimulation is absent occurs about every 1000 ms to about every 4000 ms. In some embodiments, the time period in which electrical stimulation is absent occurs about every 2000 ms to about every 3500 ms. In some embodiments, the time period in which electrical stimulation is absent occurs about every 1500 ms to about every 3000 ms. In some embodiments, the time period in which electrical stimulation is absent occurs about every 3000 ms to about every 4500 ms.

In some embodiments, one or more stimulation parameters of the systems of the present disclosure can be adjusted based on information obtained from measuring at least one neural biomarker. In some embodiments, at least one neural biomarker includes DLEP and/or oscillatory activity, and information obtained from measuring the DLEP and/or oscillatory activity indicates a need to adjust one or more stimulation parameters to treat at least one symptom in the subject. In some embodiments, the one or more stimulation parameters comprises electrode placement, electrode contact selection, stimulation parameter selection, and/or closed-loop control. In some embodiments, the one or more stimulation parameters includes stimulation pulse amplitude, stimulation pulse duration, stimulation pulse repetition rate, stimulation pulse shape, temporal pattern of stimulation pulse train, and stimulation duty cycle. In some embodiments, the system allows for the adjustment of at least one parameter based on closed-loop control of one or more biomarkers. In some embodiments, the closed-loop control is a bang-bang controller, a PI controller or a PID controller. In other embodiments, the control is a neural network (e.g., a fully convoluted neural network).

Embodiments of the present disclosure also include a system comprising a device with a processor, and a plurality of electrodes in communication with the device. The plurality of electrodes are configured to be implanted in a brain of a subject. The processor is configured to acquire, via the plurality of electrodes, a plurality of electrical signals. In some embodiments, the plurality of electrodes that acquire a plurality of electrical signals is the same plurality of electrodes that provide electrical stimulation to the brain. The processor can be configured to determine whether a biomarker is present or absent in the plurality of electrical signals, and/or to assess how a biomarker changes during the course of treatment (e.g., brain stimulation). The processor can also be configured to adjust at least one parameter based on the presence or absence of the biomarker, and/or the degree of chance in the biomarker.

In some embodiments, an electrode(s) used in the systems of the present disclosure can be one or more electrodes configured as part of the distal end of a lead or be one or more electrodes configured as part of a leadless system to apply electrical pulses to the targeted tissue region. Electrical pulses can be supplied by a pulse generator coupled to the electrode/lead. In one embodiment, the pulse generator can be implanted in a suitable location remote from the electrode/lead (e.g., in the shoulder region); however, that the pulse generator could be placed in other regions of the body or externally to the body. When implanted, at least a portion of the case or housing of the pulse generator can serve as a reference or return electrode. Alternatively, the lead can include a reference or return electrode (comprising a multipolar (such as bipolar) arrangement), or a separate reference or return electrode can be implanted or attached elsewhere on the body (comprising a monopolar arrangement).

In some embodiments, the pulse generator used in the systems of the present disclosure are electrically coupled to a plurality of electrodes, and the pulse generator includes a power source. In some embodiments, the pulse generator is implantable. The pulse generator can include stimulation generation circuitry, which can include an on-board, programmable microprocessor, which has access to and/or carries embedded code. The code expresses pre-programmed rules or algorithms under which desired electrical stimulation is generated, having desirable electrical stimulation parameters that may also be calculated by the microprocessor, and distributed to the electrode(s) on the lead. According to these programmed rules, the pulse generator directs the stimulation through the lead to the electrode(s), which serve to selectively stimulate the targeted tissue region. The code may be programmed, altered or selected by a clinician to achieve the particular physiologic response desired.

### 4. MATERIALS AND METHODS

Intraoperative experimental design. The Institutional Review Boards at Duke University and Emory University approved the study protocol. Individuals were recruited with PD undergoing DBS implantation (n=14), lead revision (n=1), implantable pulse generator (IPG) replacement (n=5), or both implantation and IPG replacement (n=1) surgery, following written informed consent (n=21, Table 1).

TABLE 1
Participant information. LFP: local field potential; DLEP: DBS local evoked potentials; Y: yes; N:
no; N/A: not available; M: male; F: female; L: left; R: right; STN: subthalamic nucleus; C: counter
electrode; AMP: stimulation amplitude; PW: pulse width; ED: blanking end delay; hr: hours; min: minutes.
Directional leads have 8 contacts arranged in a 1-3-3-1 configuration. Stimulation and recording
contacts are numbered starting at 0 with the most ventral contact (across both lead types).

| | | | | | Age - | | | | Lead type |
|---|---|---|---|---|---|---|---|---|---|
| Participant | Cohort | Motor | LFP | DLEP | Gender | Diagnosis | Procedure | Target | and model |
| 1 | Tremor | Y | Y | N | 74 - F | PD | IPG | R- | 4 ring |
| | | | | | | | replacement | STN | Medtronic |
| | | | | | | | | | 3387 |
| 2 | Tremor | Y | Y | Y | 67 - M | PD | Implant | R- | 4 ring |
| | | | | | | | | STN | Medtronic |
| | | | | | | | | | 3389 |
| 3 | Tremor | Y | Y | Y | 67 - M | PD | IPG | L- | 4 ring |
| | | | | | | | replacement | STN | Medtronic |
| | | | | | | | | | 3387 |
| 4 | Tremor | Y | Y | Y | 58 - M | PD | Implant | R- | Directional |
| | | | | | | | | STN | Boston Sci |
| | | | | | | | | | Vercise |
| | | | | | | | | | DB-2202 |
| 5 | Tremor | Y | Y | N | 52 - M | PD | Lead | L- | 4 ring |
| | | | | | | | revision | STN | Medtronic |
| | | | | | | | | | 3387 |
| 6 | Tremor | Y | Y | N | 66 - M | PD | Implant | L- | Directional |
| | | | | | | | | STN | Medtronic |
| | | | | | | | | | Sensight |
| 7 | Tremor | N | Y | Y | 73 - F | PD | Implant | R- | Directional |
| | | | | | | | | STN | Boston Sci |
| | | | | | | | | | Vercise |
| | | | | | | | | | DB-2202 |
| 8 | Tremor | Y | N/A | N/A | 65 - F | PD | Implant | R- | 4 ring |
| | | | | | | | | STN | Medtronic |
| | | | | | | | | | 3389 |
| 9 | Tremor | Y | N/A | N/A | 60 - M | PD | Implant | L- | 4 ring |
| | | | | | | | | STN | Medtronic |
| | | | | | | | | | 3389 |
| 10 | Bradykinesia | Y | Y | Y | 69 - M | PD | Implant | R- | Directional |
| | | | | | | | | STN | Boston Sci |
| | | | | | | | | | Vercise |
| | | | | | | | | | DB-2202 |
| 11 | Bradykinesia | Y | Y | Y | 63 - M | PD | IPG | R- | 4 ring |
| | | | | | | | replacement | STN | Medtronic |
| | | | | | | | | | 3389 |
| 12 | Bradykinesia | Y | Y | N | 72 - F | PD | IPG | L- | 4 ring |
| | | | | | | | replacement | STN | Medtronic |
| | | | | | | | | | 3387 |
| 13 | Bradykinesia | Y | Y | N | 61 - F | PD | Implant | R- | 4 ring |
| | | | | | | | | STN | Medtronic |
| | | | | | | | | | 3389 |
| 14 | Bradykinesia | Y | Y | Y | 63 - F | PD | Implant | R- | 4 ring |
| | | | | | | | | STN | Medtronic |
| | | | | | | | | | 3389 |
| 15 | Bradykinesia | Y | Y | Y | 62 - M | PD | Implant | R- | 4 ring |
| | | | | | | | | STN | Medtronic |
| | | | | | | | | | 3389 |
| 16 | Bradykinesia | Y | Y | Y | 53 - M | PD | Implant | R- | Directional |
| | | | | | | | | STN | Boston Sci |
| | | | | | | | | | Vercise |
| | | | | | | | | | DB-2202 |
| 17 | Bradykinesia | Y | Y | Y | 66 - M | PD | Implant | L- | 4 ring |
| | | | | | | | | STN | Medtronic |
| | | | | | | | | | 3387 |
| 18 | Bradykinesia | Y | Y | Y | 46 - M | PD | Implant | R- | 4 ring |
| | | | | | | | | STN | Medtronic |
| | | | | | | | | | 3387 |
| 19 | Bradykinesia | Y | N | N | 70 - M | PD | IPG | L- | 4 ring |
| | | | | | | | replacement | STN | Medtronic |
| | | | | | | | | | 3387 |
| 20 | Bradykinesia | Y | N | Y | 72 - M | PD | Implant | L- | 4 ring |
| | | | | | | | | STN | Medtronic |
| | | | | | | | | | 3387 |
| 21 | Bradykinesia | N | N | Y | 58 - M | PD | Implant | L- | 4 ring |
| | | | | | | | | STN | Medtronic |
| | | | | | | | | | 3387 |
| | Bradykinesia | Y | Y | Y | 63 - M | PD | IPG | L- | 4 ring |
| | | | | | | | replacement | STN | Medtronic |
| | | | | | | | | | 3387 |

| | | | | | | | Time since | |
|---|---|---|---|---|---|---|---|---|
| | | | | | | | last dose | Chronic |
| | | | | | | | of PD | clinically |
| | | | | | | | medication | determined |
| | Stimulation | Recording | AMP | PW | ED | | before | DBS |
| Participant | contacts | contacts | (V) | (us) | (us) | Gain | surgery | frequency |
| 1 | C+/1− | 0+/2− | 3 | 90 | 500 | 400 | >12 hr | 180 |
| 2 | C+/1− | 0+/2− | 3.5 | 90 | 500 | 2000 | >12 hr | missing |
| 3 | C+/2− | 1+/3− | 4 | 90 | 500 | 5000 | >12 hr | 180 |
| 4 | C+/1− | 3+/2− | 3 | 60 | 500 | 400 | >12 hr | 179 |
| 5 | C+/2− | 1+/3− | 3 | 90 | 300 | 5000 | >12 hr | 180 |
| 6 | C+/2− | 1+/3− | 1.5 | 60 | 500 | 1000 | 11 hr | 130 |
| | | | | | | | 48 min | |
| 7 | C+/1− | 2+/3− | 2.5 | 60 | 300 | 1000 | >12 hr | 149 |
| 8 | C−/2+ | N/A | 3 | 90 | N/A | N/A | >12 hr | 180 |
| 9 | C+/1− | N/A | 4 | 60 | N/A | N/A | >12 hr | 185 |
| 10 | C+/1− | 2+/3− | 3 | 60 | 500 | 1000 | >12 hr | 130 |
| 11 | C+/2− | 1+/3− | 2.5 | 80 | 500 | 400 | >12 hr | 150 |
| 12 | C+/1− | 0+/2− | 3.6 | 80 | 200 | 2000 | >12 hr | 180 |
| 13 | C+/1− | 0+/2− | 2-2.5 | 90 | 300 | 2000 | >12 hr | 185 |
| 14 | C+/1− | 2+/0− | 2.5 | 90 | 300 | 1000 | >12 hr | 160 |
| 15 | C+/1− | 0+/2− | 1.25 | 60 | 200 | 1000 | >12 hr | 130 |
| 16 | C+/1− | 2+/3− | 2 | 60 | 300 | 200 | >12 hr | 130 |
| 17 | missing | missing | 3 | 60 | 100 | 500 (assumed | missing | 180 |
| | | | | | | default) | | |
| 18 | C+/1− | 0+/2− | 2.5 | 90 | 200 | 2000 | >12 hr | 150 |
| 19 | C+/1− | 0+/2− | 2.9 | 90 | 500 | 1000 | >12 hr | 180 |
| 20 | C+/2− | 1+/3− | 2.5 | 90 | 500 | 10000 | >12 hr | 180 |
| 21 | C+/1− | 0+/2− | 2.5 | 90 | 500 | 2000 | >12 hr | 180 |
| | C+/2− | 3+/1− | 2 | 60 | 300 | 1000 | >12 hr | 160 |

Previous reports investigated beta bursts in persons with PD that included data from a small subset of these participants (n=6), but only beta burst characteristics during high frequency DBS and Off stimulation were analyzed (the effect of DBS on motor symptoms was not analyzed, nor was any temporally non-regular pattern of DBS evaluated). Most participants withdrew from PD medication for at least 12 hours before surgery (one participant withdrew from medication for 11 hours and 48 minutes before surgery).

Intraoperative experiments were conducted, simultaneously delivering DBS and recording oscillatory activity from the STN of each participant. For implants and lead revisions, signals were recorded from the new DBS lead after it was implanted in its final location, following micro-electrode recordings (MER) and neurologist testing. For IPG replacements for depleted batteries, signals were recorded directly from the DBS extension after the old pulse generator was removed and before the new one was placed. Motor symptoms were also recorded from the contralateral side before and during DBS according to each participant's dominant symptom (tremor, n=9, or bradykinesia, n=12). During preoperative screening, participants who presented a considerable amount of upper limb tremor that was deemed to interfere with the intraoperative bradykinesia motor task (clicking of a computer mouse) were assigned to the tremor-dominant cohort, and all other participants were assigned to the bradykinesia-dominant cohort.

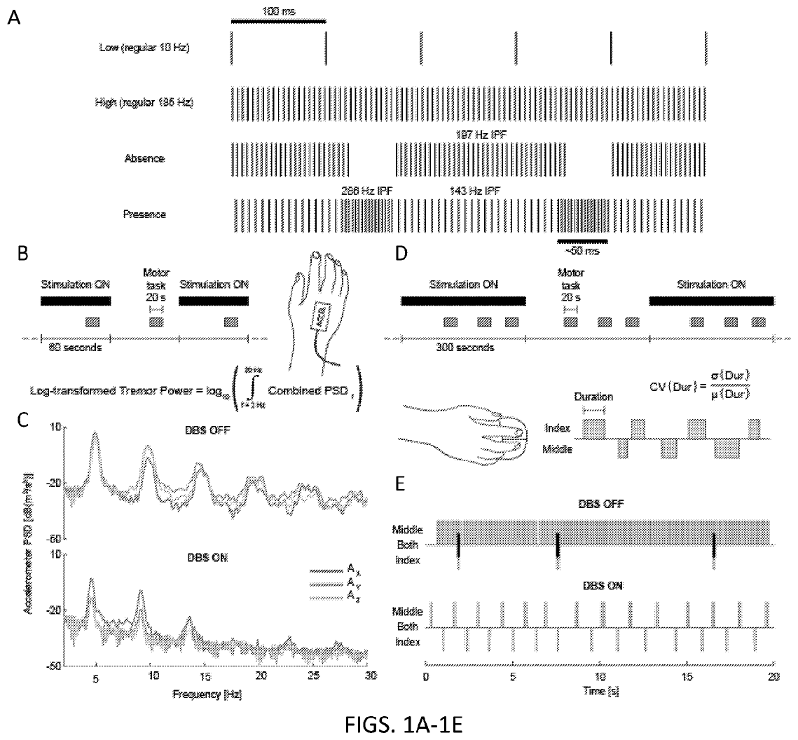

The effects of four DBS patterns on symptoms and LFP were measured (FIG. 1A). Two patterns consisted of constant rate stimulation, 10 Hz (Low) and 185 Hz (High), which prior studies indicate are ineffective and effective, respectively, in treating the motor symptoms of PD. The remaining patterns were temporally non-regular patterns with geometric mean of 185 Hz (Absence and Presence). Both patterns were periodic and characterized by the absence or presence of short bursts of pulses. Absence consisted of 197 Hz stimulation with ˜50 ms gaps of stimulation occurring every ˜200 ms. Presence consisted of 143 Hz stimulation with ˜50 ms bursts of 286 Hz stimulation occurring every ˜200 ms. Stimulation amplitude and pulse width were kept constant across all stimulation patterns for each participant, but these parameters slightly varied between participants, as determined by the attending neurologist for implantation and lead revision surgeries or by existing DBS parameters for IPG replacement surgeries (Table 1).

Absence and Presence are two patterns that were designed to test which stimulus features of temporally non-regular DBS were responsible for reduced tremor suppression, in a separate group of tremor patients with DBS of the ventral intermediate (VIM) nucleus of the thalamus. Pauses in stimulation (such as those in Absence) reduced the efficacy of VIM DBS when treating tremor. In contrast to the tremor patients, these same patterns were also tested in participants with PD, during DBS of either the STN or the globus pallidus internal (GPi) segment. The results indicated that these patterns were more effective in reducing bradykinesia in persons with PD than fixed rate 185 Hz DBS, and a computational model predicted that this increased efficacy was due to increased suppression of pathological beta activity in the basal ganglia. In the present study, experiments were conducted to test these model predictions, as well as expand the analysis to include both tremor and bradykinesia in PD.

The Low and High stimulation patterns were chosen as negative and positive controls, respectively, since DBS is known to be effective only at high frequencies (over 100 Hz, typically in the 130-185 Hz range). 185 Hz was chosen for High because it has the same geometric mean frequency of 185 Hz as Absence and Presence, thus being the most suitable positive control for comparison. Since the most common clinical DBS frequency is 130 Hz, the chronic, clinically determined DBS frequency was reported for all participants in Table 1 for comparison. The clinical DBS frequency of most participants (81%) was higher than 130 Hz and spanned a range of 130 to 185 Hz. The mean of the chronic, clinically determined DBS frequency for all participants was 165 Hz±21 Hz (standard deviation).

Tremor power was measured (n=9) with a hand-mounted accelerometer and quantified it using all 3 accelerometer axis components (FIG. 1B). During tremor trials, participants remained at rest. Bradykinesia was measured (n=13) using an alternating finger tapping task (FIG. 1D) that is correlated to the motor section of the Unified Parkinson's Disease Rating Scale (UPDRS III). During bradykinesia trials, the mouse was placed on the bed at the side of the participants, so they could easily place their hands on the mouse.

Different patterns of stimulation were delivered in a randomized block design, and trials alternated between Off and On stimulation to allow the effects of stimulation to washout before the next trial. LFPs were recorded throughout the duration of a trial. Tremor trials were 60 s long and motor symptoms were evaluated during one 20 s epoch at 30 s after trial onset (FIG. 1B). Bradykinesia trials were 300 s long and alternating finger tapping was evaluated during three separate epochs of 20 s each at 90, 210, and 260 s after trial onset (FIG. 1D). The trial length was increased for bradykinesia because the effect of DBS on bradykinesia is known to develop over longer timescales, whereas the effect of DBS on tremor is almost immediate. Each stimulation pattern was tested once to complete an experimental block. Bradykinesia- and tremor-dominant participants completed one or three blocks, respectively. Bradykinesia-dominant participants were less likely to complete one full block compared to tremor-dominant participants (block duration was 40 minutes for bradykinesia and 8 minutes for tremor). Therefore, for bradykinesia-dominant participants only, Low stimulation was delivered during the last trial of the block.

LFPs were recorded differentially using two contacts that flanked the monopolar stimulation contact (Table 1). For directional leads (n=5), the two contacts on the same ring as the stimulation contact were used for recording to match more closely the impedances of the recording contacts for optimal artifact rejection. The lead implant cannula or a retractor placed in the IPG pocket (depending on the surgery) served as reference (ground). The recording instrumentation was modified from a previously published design. The LFP of some of the initial participants was recorded using the original design, and used the updated design for the rest of the participants. To increase electrical safety, anti-series current-limiting (0.1 mA) diodes (E-101, SEMITEC) and series capacitors (20 μF, F461-464, KEMET) were placed between the DBS lead and recording instrumentation. The signal passed through two battery-powered amplifiers (SR560, Stanford Research Systems). Antiparallel diode clamps (BAT 63-02V, Infineon Technologies) were used between the two amplification stages to limit stimulation artifact amplitude (±190 mV). The first amplifier was DC-coupled and set for differential recording, and the second amplifier was AC-coupled and set for single-ended recording. The gain of each amplifier was set to either 20 or 50 V/V (Table 1). Additionally, the second amplifier had a low pass filter at 10 kHz and a high pass filter at 0.1 Hz. The sampling rate was either 50 or 100 kHz. The second amplifier was blanked 20 us before, during, and for 100-500 us after each stimulation pulse (Table 1). The LFP was then digitized by a NIDAQ 6216 (National Instruments, Austin TX) with 16-bit resolution over a 2V range.

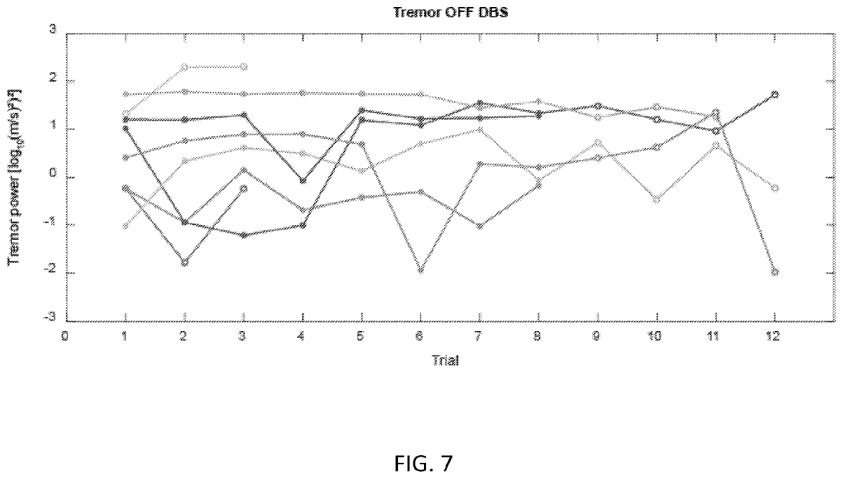

Motor symptom quantification. Tremor was quantified as the log ₁₀-transform of the combined power spectral density of all 3 accelerometer axis components between 2 and 20 Hz (FIG. 1B). Since there was no worsening of tremor over time during Off trials (FIG. 7), data from multiple experimental blocks were averaged (up to 3). FIG. 1C shows an example of how the accelerometer detected changes in tremor during DBS Off and On.

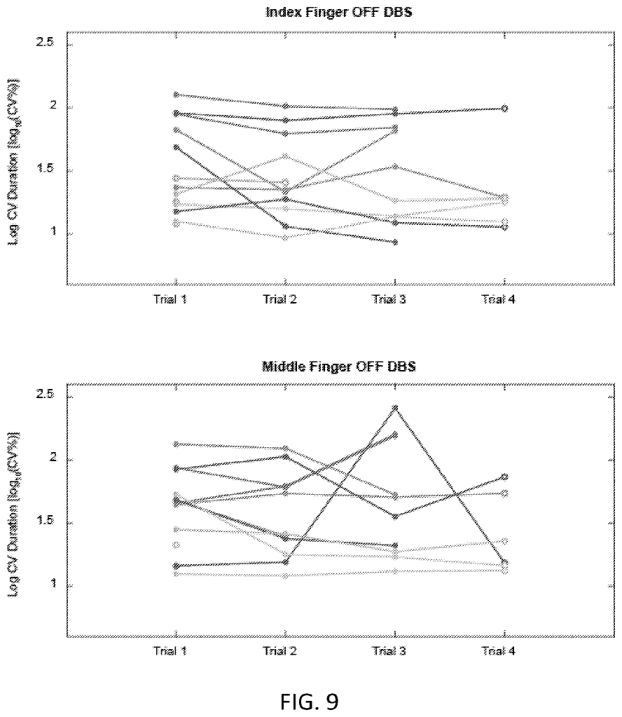

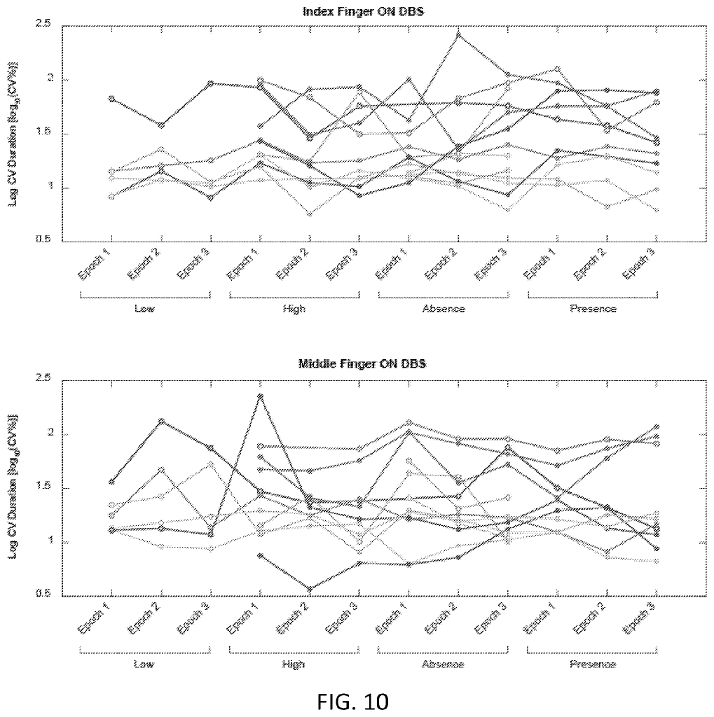

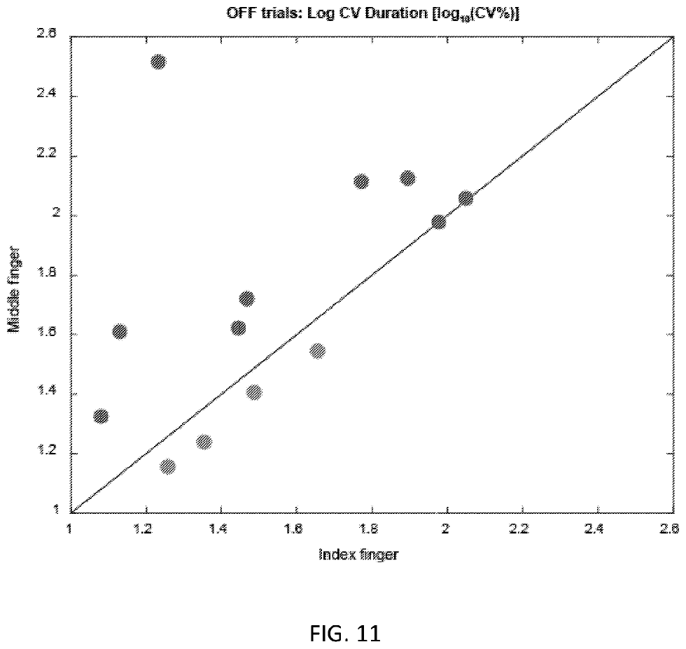

Bradykinesia was quantified using an alternating finger tapping task and analyzed the index and middle fingers separately. For each finger, the coefficient of variation (CV, standard deviation divided by the mean) of the click durations was calculated (FIG. 1D). The CV was then log ₁₀-transformed, and the resulting value was correlated with bradykinesia. Since there was no worsening of bradykinesia over time during Off trials (FIG. 9), click durations were pooled during Off trials before CV calculation. The effect of time since stimulation onset on bradykinesia was not detected (FIG. 10), and therefore click durations were pooled the during the three epochs of a trial before CV calculation. Even though epoch did not have a statistically significant effect on bradykinesia, a trend was present, with the first epoch showing more severe bradykinesia than the subsequent two epochs (least squares mean of epoch 1 was greater than that of epochs 2 and 3). This effect was not pronounced enough in this data set to yield statistical significance, but it is consistent with the literature that the effects of DBS on bradykinesia occur on longer timescales. The log ₁₀CV was calculated for each participant's index and middle fingers during Off, and selected the worst-performing finger for all subsequent analyses to avoid ceiling effects (FIG. 11). FIG. 1E shows an example of how the alternating finger tapping task detected changes in bradykinesia during DBS Off and On.

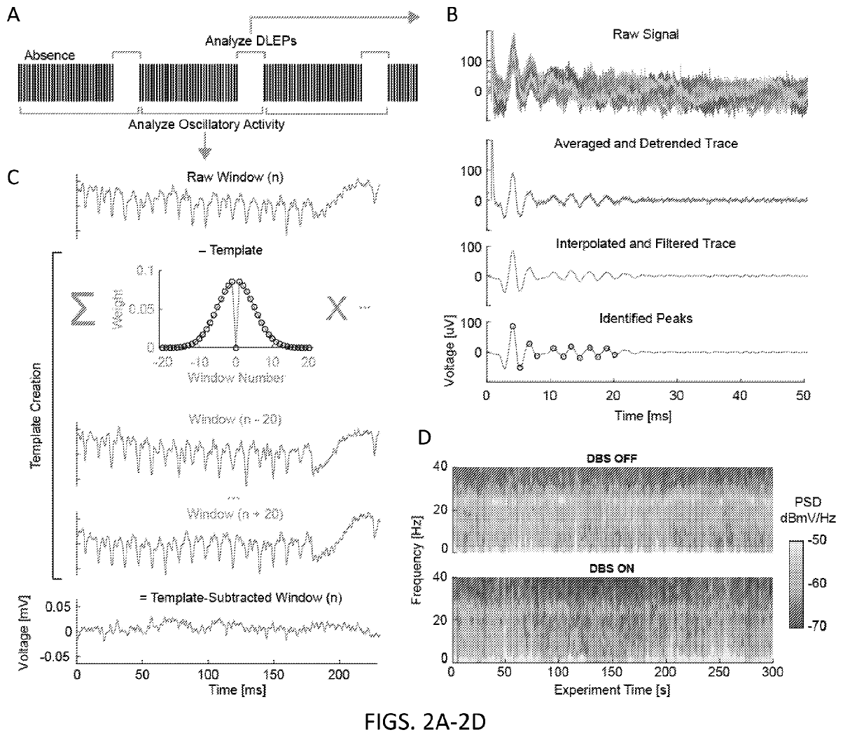

Neural signal analysis. Both oscillatory activity and DLEPs were calculated from the LFP (FIG. 2A). Signal processing was conducted in MATLAB R2019b (MathWorks, Natick, MA).

To quantify oscillatory activity in the STN, template subtraction was used to remove residual stimulation artifact from the LFP signal (FIG. 2C). Template subtraction consisted of three steps. First, an appropriate window length was selected for each stimulation pattern. The shortest possible window length was given by the shortest time period of unique stimulation features found in each stimulation pattern. For example, the shortest possible window length for Absence consisted of one section of 197 Hz stimulation and one section of no stimulation. For Absence and Presence, the window length was approximately 200 ms. For Low and High, even though the window length could be shorter, a window length of approximately 200 ms was selected for consistency. To ensure all files were processed equally, template subtraction was also conducted on Off trials, with a window length of 200 ms, even though there were no stimulation artifacts. Second, for a given window of the LFP signal, a template was created by calculating a weighted Gaussian average of the 20 windows preceding and the 20 windows following the current window. The current LFP window was given a weight of zero when creating the template. Lastly, the template was subtracted from the current LFP window. This procedure was repeated for every consecutive LFP window.

Template subtraction relies on the assumption that the stimulation artifact is constant over time periods longer than the physiological frequency ranges of interest. While the environment around a DBS lead does change over time, as indicated by changes in electrode impedance, this happens over the course of months and years. Therefore, any changes to the local environment surrounding the DBS lead, which could potentially affect the stimulation artifact, occur over time periods much longer than the physiological frequency ranges of interest.

Template subtraction was necessary because the two temporally non-regular stimulation patterns that were tested introduced artifacts in the frequency bands of interest. The Absence stimulation pattern consisted of 50 ms gaps repeating every 200 ms, resulting in artifacts at 20 Hz and 5 Hz, respectively. These artifacts contaminate two of the frequency bands of interest: beta (13-35 Hz) and theta (4-7 Hz). The Presence stimulation pattern also repeated every 200 ms and thus presented similar challenges. The Low stimulation pattern contained artifacts at 10 Hz, which contaminated the alpha band (8-12 Hz). Off and High stimulation patterns did not have these same issues, but template subtraction was conducted on those patterns as well for consistency.

The LFP was further processed using a bandpass 4^{th} order non-causal Butterworth filter and down-sampling to 500 Hz. LFP power was quantified during the motor task period using Welch's power spectral density (PSD) estimate for the theta (4-7 Hz), alpha (8-12 Hz), and beta (13-35 Hz) bands. In the bradykinesia-dominant cohort, beta power was quantified, which is correlated with bradykinesia and rigidity. In the tremor-dominant cohort, theta, alpha, and beta power were quantified. Previous studies demonstrated that STN theta power and tremor are correlated, while STN beta power and tremor are negatively correlated. Another study found that alpha power in the cortex was reduced during STN DBS.

The input signal to the Welch PSD estimate was the LFP recorded during the 20 seconds long motor task. A Hamming window was used to obtain eight segments of the input signal with 50% overlap between segments. For a given frequency range, the integrated power of the PSD was calculated within the frequency range of interest. FIG. 2D shows spectrograms obtained after template subtraction during DBS Off and On, and the spectrograms reveal modulation of both low (<20 Hz) and high (>20 Hz) beta sub-bands during DBS.

To analyze DLEPs, the raw LFP signal was used (FIG. 2B) as template subtraction removes DLEPs from the signal. For visualization purposes, the mean value of the LFP was subtracted during each inter-pulse interval (IPI) after the stimulation artifact (>2.5 ms). Signals were averaged within the IPIs in advancing windows of 2 s (no overlap) across the entire trial. Each averaged trace was detrended using a moving average (3 ms) to remove stimulation artifacts that persisted for longer than a couple of ms. The resulting traces were filtered using a 1 kHz lowpass 3^{rd} order non-causal Butterworth filter, and a peak-detecting algorithm (which used MATLAB's findpeaks) identified the positive and negative peaks in each averaged DLEP trace. For Low and High, DLEPs were analyzed across all IPIs. For Absence, DLEPs were analyzed only across the ˜50 ms gaps (FIG. 2A), while for Presence only during the 143 Hz stimulation periods. Three DLEP metrics were quantified: number of periods, and the amplitude and latency of the first positive peak (P1). As a result of the short IPIs, only P1 was visible during High and Presence. Therefore, these two stimulation patterns were not included in the statistical analyses involving the number of periods and P1 amplitude (calculated as the distance between the first positive and first negative peaks of the DLEPs). P1 latency was quantified for all four stimulation patterns. For Absence, the signal-to-noise (SNR) ratio was calculated as the ratio of P1 amplitude in the first trace to the mean noise floor of the entire trial (twice the standard deviation of the 5 ms window that ends 1 ms before the next pulse, when DLEPs were no longer detectable).

Statistical methods. All statistical analyses were conducted in JMP Pro Version 15 (SAS Institute Inc., Cary, NC) with the Full Factorial Repeated Measures ANOVA add-in. A total of eight global ANOVAs were conducted. An overview of all statistical tests is provided herein.

Interparticipant variability was pronounced and therefore assigning participant as a random factor was crucial to reveal trends in the data. To protect from multiple comparisons, global ANOVAs were run and the data was only subdivided for further analyses when the relevant interaction terms between factors were statistically significant (alpha=0.05). All reported p-values are protected from multiple comparisons.

Due to missing values of repeated measures, some data were initially excluded from global ANOVAs, and subsequently added back in when subdividing by repeated measure. Statistical software usually deals with missing data for repeated measures by Winsorizing (interpolating or extrapolating) the missing data before running statistical tests. Additionally, different software may use different methods of Winsorizing data without explicitly stating what method was used, making it very challenging for a third party to replicate the same results. Data in the global ANOVA was excluded and was added back in when subdividing by repeated measure to avoid using invented numbers and maintain research integrity by facilitating reproducibility of the statistical methods. This approach is more conservative than using a linear model and Winsorizing missing data points because it reduces the overall n (and hence reduces statistical power), so that when a statistically significant difference was found, it was deemed to be “real,” i.e., not caused by inserting Winsorized data.

The raw data were log-normally distributed and therefore all statistical tests were conducted on log ₁₀-transformed data (with one exception). When subdividing the DLEP data by repeated measure, zero-valued data was added back into the number of periods subgroup. In this instance, the square root was used transform instead of the log ₁₀-transform. Zero data cannot be log ₁₀-transformed, and adding a constant to the data before log ₁₀-transformation is not appropriate, since the selection of the constant is arbitrary and can distort the distributions and greatly impact the results. The analysis of the DLEP number of periods was repeated twice: excluding the zero data and using the log ₁₀-transform, and again including the zero data and using the square root transform. The results did not change (p values from the square root transform analysis was reported in the main text). Detailed descriptions (within- and between-subject factors) and results (degrees of freedom, F ratios, p values and effect sizes) of all statistical tests is provided herein.

To confirm that the transformed data were normally distributed, Shapiro-Wilk tests were conducted on all groups before conducting global ANOVAs. Out of 42 Shapiro-Wilk tests, 39 were not significant, confirming the data in those groups were normally distributed; the remaining 3 were significant and therefore not normally distributed. All Shapiro-Wilk tests are provided herein. Given that most groups were normally distributed, parametric analysis was employed. Non-parametric tests do not allow discussion of the magnitude of effect, which requires parametric analysis, and therefore parametric tests are preferable.

When analyzing motor symptoms and oscillatory activity in the LFP, the tremor- and bradykinesia-dominant cohorts were kept separate. In these statistical tests, motor symptoms and LFP power were labeled as a repeated measure (within subject). Stimulation pattern was also labeled as a repeated measure, since each participant received multiple stimulation patterns. The data contained missing values due to participants withdrawing from the protocol before completion of an experimental block. To avoid discarding data from incomplete blocks or imputing the missing values, global ANOVAs were first conducted only on completed blocks and then the incomplete blocks were added back in when subdividing the data, since the missing values were then no longer a problem. The bradykinesia-dominant cohort had more missing data than the tremor-dominant cohort. Therefore, to maximize the number of participants included in the statistical analysis, Low was excluded from the bradykinesia-dominant cohort when evaluating oscillatory activity and motor symptom. Data that was fully excluded from analysis was plotted as open circles across all figures.

When analyzing DLEPs, the tremor- and bradykinesia-dominant cohorts were combined. Since the original experimental design did not include characterization of DLEPs, it was not appropriate to correlate motor symptoms with DLEP evolution. Nonetheless, bradykinesia and DLEP characteristics were correlated at each of the three time points of motor symptom evaluation (not shown here). However, both DLEPs and bradykinesia had reached their steady-state values by 90 s (the first time point of motor symptom evaluation) and thus no correlations were observed. This was consistent with an earlier analysis that did not detect an effect of time since stimulation onset (90, 210, and 260 s) on bradykinesia (FIG. 10). Motor symptoms were thus excluded from this retrospective analysis, allowing for combining the tremor- and bradykinesia-dominant cohorts. In these statistical tests, stimulation pattern was not considered to be a repeated measure. Since trial duration varied between tremor- and bradykinesia-dominant cohorts (60 and 300 s, respectively), participants in the tremor-dominant cohort had missing DLEP data from 60 to 300 s. When conducting statistical analyses on the DLEP data set, time since stimulation onset was labeled as a repeated measure. Since repeated measures ANOVA cannot handle missing data, DLEP data beyond 60 s was excluded from statistical analyses and plotted them as open circles across all figures.

Participant 21 enrolled in the study twice (during DBS implantation and IPG replacement, approximately 5 years apart). Given that the stimulation and recording contacts used during each surgery were different, and that PD had progressed, it was determined as not appropriate to average data across both surgeries. When analyzing motor symptoms and oscillatory activity in the LFP (where stimulation pattern was a repeated measure), data from only one surgery were included. The IPG replacement data was included because it contained all four stimulation patterns (whereas the implantation data contained only one stimulation pattern). When analyzing DLEPs (where stimulation pattern was not a repeated measure), data from both implantation and IPG replacement surgeries were included.

Principal component analysis (PCA). PCA was conducted on the beta power and bradykinesia data using JMP Pro Version 15 (SAS Institute Inc., Cary, NC). Individual participants were not explicitly represented in the PCA, since PCA is not appropriate to use on categorical values. Since PCA was ran on only two continuous variables (beta power and bradykinesia), the PCA output only two principal components. From JMP, the two formulas were extracted to map each data point from Cartesian space (where the x and y axes represent beta power and bradykinesia, respectively) to principal component space. Next, the second principal component (PC2) was set to zero and the data points were mapped back to Cartesian space using the JMP formulas. The resulting Cartesian coordinates represented the variability of the data that was captured by the first principal component (PC1). This PCA approach was used to remove the variability of bradykinesia that was due to PC1 before conducting a correlation between beta power and bradykinesia.

Provided below are comprehensive statistical descriptions (within- and between-subject factors) and results (degrees of freedom, effect sizes, F ratios and p values) of all analyses, labeled by figure. All statistical analyses were conducted in JMP Pro Version 15 (SAS Institute Inc., Cary, NC) with the Full Factorial Repeated Measures ANOVA add-in.

In Tables 2 and 3, data and missing values for the tremor and bradykinesia cohorts are provided. Certain values were supplemented for the following reasons: 2 participants from the tremor cohort did not have LFP data (these data were collected before the instrumentation to record LFPs intraoperatively was built); 1 participant from the tremor cohort did not have tremor data (the accelerometer was not connected properly to the computer); and 7 participants from the bradykinesia cohort withdrew from the study before a full experimental block was completed: 4 participants only received High, Absence, and Presence stimulation; 1 participant only received High and Absence stimulation; 1 participant only received Absence stimulation; The 1 participant that was enrolled twice (implant and battery change) only received Absence during DBS implant, and received all four stimulation patterns during battery change.

TABLE 2
Tremor cohort. Units for Log theta/alpha/beta are
log₁₀(mV²). Units for Log motor are log₁₀[(m/s²)²].

| | Off | | | | Low | | | | High | |
|---|---|---|---|---|---|---|---|---|---|---|
| | Log | Log | Log | Log | Log | Log | Log | Log | Log | Log |
| ID | theta | alpha | beta | motor | theta | alpha | beta | motor | theta | alpha |
| 1 | −5.02 | −4.88 | −4.36 | −0.31 | −4.94 | −4.85 | −4.35 | 0.21 | −5.12 | −4.90 |
| 2 | −4.79 | −4.96 | −4.96 | 1.23 | −4.68 | −4.90 | −4.77 | 1.08 | −5.28 | −5.32 |
| 3 | −5.10 | −5.40 | −5.00 | 1.63 | −5.14 | −5.38 | −5.00 | 1.51 | −5.06 | −5.32 |
| 4 | −3.00 | −3.70 | −3.40 | 0.71 | −2.69 | −3.20 | −3.40 | 0.98 | −3.45 | −3.97 |
| 5 | −5.89 | −5.92 | −5.94 | 0.49 | −5.87 | −5.98 | −6.03 | 0.44 | −5.88 | −5.99 |
| 6 | −5.02 | −5.20 | −5.01 | 1.21 | −5.60 | −5.94 | −5.53 | 1.35 | −4.70 | −5.08 |
| 7 | −5.17 | −5.43 | −5.01 | missing | −5.30 | −5.45 | −5.34 | missing | −5.42 | −5.72 |
| 8 | missing | missing | missing | −0.40 | missing | missing | missing | missing | missing | missing |
| 9 | missing | missing | missing | 2.14 | missing | missing | missing | 2.20 | missing | missing |
| | High | | Absence | | | | Presence | | | |
| | Log | Log | Log | Log | Log | Log | Log | Log | Log | Log |
| ID | beta | motor | theta | alpha | beta | motor | theta | alpha | beta | motor |
| 1 | −4.36 | −1.65 | −5.06 | −4.94 | −4.41 | −1.71 | −5.07 | −5.01 | −4.36 | −1.68 |
| 2 | −5.65 | 0.75 | −4.78 | −5.02 | −5.02 | 1.28 | −5.09 | −5.39 | −5.62 | 1.16 |
| 3 | −5.08 | −1.66 | −5.06 | −5.37 | −5.11 | −1.51 | −5.15 | −5.42 | −5.12 | −1.68 |
| 4 | −3.49 | −1.94 | −2.94 | −3.64 | −3.47 | 0.06 | −3.00 | −3.75 | −3.47 | 0.58 |
| 5 | −6.06 | 0.09 | −5.81 | −6.03 | −6.00 | 0.35 | −5.82 | −5.99 | −6.02 | 0.25 |
| 6 | −4.67 | 0.98 | −5.15 | −5.46 | −5.23 | −1.77 | −5.06 | −5.33 | −4.95 | 1.16 |
| 7 | −5.20 | missing | −5.36 | −5.42 | −5.03 | missing | −5.35 | −5.61 | −5.08 | missing |
| 8 | missing | −1.76 | missing | missing | missing | −1.77 | missing | missing | missing | −1.75 |
| 9 | missing | 1.88 | missing | missing | missing | missing | missing | missing | missing | 2.05 |

TABLE 3
Bradykinesia cohort. Units for Log beta are
log₁₀(mV²). Units for Log motor are log₁₀(CV %).

| | Off | | Low | | High | | Absence | | Presence | |
|---|---|---|---|---|---|---|---|---|---|---|
| | Log | Log | Log | Log | Log | Log | Log | Log | Log | Log |
| ID | beta | motor | beta | motor | beta | motor | beta | motor | beta | motor |
| 10 | −5.05 | 2.06 | missing | missing | −5.42 | 1.99 | −5.45 | 1.92 | −5.36 | 1.98 |
| 11 | −3.85 | 1.72 | −3.79 | 1.53 | −3.96 | 1.40 | −3.88 | 1.27 | −3.82 | 1.10 |
| 12 | −4.65 | 1.35 | −4.66 | 1.06 | −4.57 | 1.12 | −4.61 | 1.14 | −4.69 | 1.09 |
| 13 | −4.98 | 1.49 | −5.00 | 1.28 | −5.42 | 1.17 | −5.26 | 1.21 | −5.13 | 1.19 |
| 14 | −5.47 | 2.11 | missing | missing | −5.63 | 1.95 | −5.69 | 1.99 | −5.58 | 1.86 |
| 15 | −5.07 | 1.98 | −5.19 | 1.98 | −5.31 | 1.43 | −5.27 | 1.79 | −5.22 | 1.44 |
| 16 | −4.40 | 2.52 | −4.63 | 1.11 | −4.85 | 2.49 | −4.75 | 1.20 | −4.78 | 1.29 |
| 17 | −4.41 | 1.66 | missing | missing | −4.74 | 1.42 | −4.75 | 1.40 | −4.74 | 1.90 |
| 18 | −4.91 | 2.12 | missing | missing | −5.55 | 1.72 | −5.54 | 1.94 | −5.59 | 2.01 |
| 19 | −4.73 | 1.62 | missing | missing | −4.53 | 1.26 | −4.41 | 1.58 | missing | missing |
| 20 | −5.21 | 1.32 | missing | missing | missing | missing | −5.65 | 1.25 | missing | missing |
| 21 | −4.79 | 1.26 | missing | missing | missing | missing | −5.55 | 1.14 | missing | missing |
| (implant) | | | | | | | | | | |
| 21 | −3.83 | 1.61 | −3.88 | 1.18 | −3.84 | 1.24 | −3.88 | 1.24 | −3.79 | 1.29 |
| (battery | | | | | | | | | | |
| change) | | | | | | | | | | |

In Tables 4 and 5, data and missing values for the DLEP cohorts are provided. Certain values were supplemented for the following reasons: not all participants received all stimulation patterns (see earlier explanation); DLEPs were not visible in every recording for every patient (see Table 1 for a list of which participants showed DLEPs in at least one trial); trials of the tremor-dominant cohort were only 60 seconds (as opposed to 300 seconds like the trials of the bradykinesia-dominant cohort), therefore, these trials had missing values between 60 and 300 seconds; and if the number of DLEP periods was zero, the P1 latency could not be calculated, therefore, a given trial may have data for the number of periods, but missing values for P1 latency.

TABLE 4
DLEP P1 latency cohort. Units for P1 latency are seconds.

| | Low | | | High | | | Absence | | | Presence | | |
|---|---|---|---|---|---|---|---|---|---|---|---|---|
| ID | 1 s | 51 s | 291 s | 1 s | 51 s | 291 s | 1 s | 51 s | 291 s | 1 s | 51 s | 291 s |
| 2 | 0.00418 | 0.0041433 | missing | 0.0032133 | missing | missing | 0.0031567 | 0.0044633 | missing | 0.00369 | 0.0049633 | missing |
| 3 | 0.00584 | 0.00583 | missing | 0.0031033 | missing | missing | 0.0058833 | 0.0061367 | missing | 0.0029433 | 0.0031233 | missing |
| 4 | 0.0063933 | 0.0064067 | missing | 0.00314 | 0.00374 | missing | 0.0031267 | missing | missing | 0.00314 | 0.00404 | missing |
| 7 | 0.00441 | 0.0044 | missing | 0.00375 | 0.00423 | missing | 0.00371 | 0.00444 | missing | 0.00396 | 0.00499 | missing |
| 10 | missing | missing | missing | 0.00328 | 0.00388 | 0.00407 | 0.00322 | 0.0037 | missing | 0.00348 | 0.00535 | missing |
| 11 | 0.00418 | missing | missing | 0.0038 | 0.00429 | missing | 0.0038 | missing | missing | 0.00382 | 0.00419 | 0.00422 |
| 14 | missing | missing | missing | 0.00354 | missing | missing | 0.0035 | 0.00427 | missing | 0.00394 | 0.00514 | missing |
| 15 | 0.00562 | 0.00553 | 0.00552 | missing | missing | missing | 0.00514 | 0.00539 | 0.00545 | 0.00534 | 0.00594 | 0.00587 |
| 16 | 0.00406 | 0.00407 | 0.0041 | 0.00366 | 0.00426 | 0.00426 | 0.00366 | 0.00433 | 0.00418 | 0.00388 | 0.00445 | 0.0046 |
| 17 | missing | missing | missing | 0.00419 | 0.00451 | missing | 0.00414 | 0.00441 | missing | 0.00427 | 0.00519 | 0.00534 |
| 18 | missing | missing | missing | 0.00352 | missing | missing | 0.00356 | 0.00451 | missing | 0.00375 | 0.005005 | 0.005195 |
| 20 | missing | missing | missing | missing | missing | missing | 0.00407 | 0.00451 | 0.0046 | missing | missing | missing |
| 21 (implant) | missing | missing | missing | missing | missing | missing | 0.00361 | 0.004715 | 0.004535 | missing | missing | missing |
| 21 (battery | 0.0042 | 0.00416 | 0.00422 | 0.00422 | missing | missing | 0.0043 | 0.00392 | missing | 0.00432 | 0.00426 | 0.00418 |
| change) | | | | | | | | | | | | |

TABLE 5
DLEP amplitude and n periods cohort. Units for N periods
are counts. Units for P1 amplitude are volts.

| | Low | | | | | |
|---|---|---|---|---|---|---|
| | N periods | | | P1 amplitude | | |
| ID | 1 s | 51 s | 291 s | 1 s | 51 s | 291 s |
| 2 | 5 | 5.3333333 | missing | 0.0007767 | 0.0007185 | missing |
| 3 | 1 | 1 | missing | 2.17E−05 | 2.92E−05 | missing |
| 4 | 1.6666667 | 1.3333333 | missing | 9.27E−05 | 0.0001079 | missing |
| 7 | 4.5 | 4 | missing | 0.0002318 | 0.000245 | missing |
| 10 | missing | missing | missing | missing | missing | missing |
| 11 | 1 | 0 | 0 | 2.57E−05 | missing | missing |
| 14 | missing | missing | missing | missing | missing | missing |
| 15 | 3 | 4 | 3.5 | 3.46E−05 | 3.20E−05 | 3.49E−05 |
| 16 | 3 | 2.5 | 3 | 0.0002103 | 0.0002115 | 0.0002895 |
| 17 | missing | missing | missing | missing | missing | missing |
| 18 | missing | missing | missing | missing | missing | missing |
| 20 | missing | missing | missing | missing | missing | missing |
| 21 (implant) | missing | missing | missing | missing | missing | missing |
| 21 (battery | 1 | 0.75 | 1 | 1.84E−05 | missing | 1.90E−05 |
| change) | | | | | | |

| Absence | |
|---|---|
| N periods | P1 amplitude |

| ID | 1 s | 51 s | 291 s | 1 s | 51 s | 291 s |
|---|---|---|---|---|---|---|
| 2 | 11 | 4.3333333 | missing | 0.0004171 | 2.96E−05 | missing |
| 3 | 2 | 1.3333333 | missing | 5.90E−05 | 2.51E−05 | missing |
| 4 | 2.3333333 | 0 | missing | 0.0001213 | missing | missing |
| 7 | 3.5 | 4 | missing | 0.0001474 | 7.26E−05 | missing |
| 10 | 4 | 2.5 | 0 | 4.30E−05 | 1.94E−05 | missing |
| 11 | 3 | 0 | 0 | 8.55E−05 | missing | missing |
| 14 | 6 | 1 | 0 | 5.72E−05 | 1.45E−05 | missing |
| 15 | 7 | 4 | 3 | 4.33E−05 | 3.58E−05 | 2.20E−05 |
| 16 | 6 | 5 | 4 | 0.0006553 | 0.0001961 | 0.0001399 |
| 17 | 3 | 1.5 | 0 | 0.0002211 | 7.00E−05 | missing |
| 18 | 16 | 5 | 0 | 0.0001349 | 3.91E−05 | missing |
| 20 | 6 | 4.5 | 4 | 0.0001375 | 5.63E−05 | 5.22E−05 |
| 21 (implant) | 17 | 8 | 5 | 0.0008236 | 0.0001283 | 7.01E−05 |
| 21 (battery | 1 | 0.5 | 0 | 1.96E−05 | missing | missing |
| change) | | | | | | |

A total of eight global ANOVAs were performed. Different global ANOVAs were necessary for different experiments and datasets. In Table 6, an overview of all global ANOVAs is provided, labeled by figure. In Tables 7-8, all statistical results are reported.

TABLE 6
Overview of all global ANOVAs.

| Figure | Data | Test | Within-subject factors | Between-subject factors |
|---|---|---|---|---|
| 3 | Tremor cohort (motor and | Global | Stimulation (5 levels: | N/A |
| | LFP data) | ANOVA | Off, Low, High, Absence, | |
| | | | Presence) | |
| | | | Measure (2 levels: | |
| | | | Theta Power, Tremor) | |
| 4 | Bradykinesia cohort (motor | Global | Stimulation (4 levels: Off, | N/A |
| | and LFP data) | ANOVA | High, Absence, Presence) | |
| | | | Measure (2 levels: | |
| | | | Beta Power, Bradykinesia) | |
| 6CD | DLEP cohort (N periods and | Global | Time (2 levels: 1 s, 51 s) | Stimulation (2 levels: Low, |
| | P1 amplitude) | ANOVA | Measure (2 levels: Number | Absence) |
| | | | of Periods, P1 Amplitude) | |
| 6E | DLEP cohort (P1 latency) | Global | Time (2 levels: 1 s, 51 s) | Stimulation (4 levels: Low, |
| | | ANOVA | | High, Absence, Presence) |
| 7 | Tremor cohort (averaging | Global | Trial (8 levels: Trials | N/A |
| | off trials) | ANOVA | 1 through 8) | |
| 8 | Tremor cohort (LFP data | Global | Stimulation (4 levels: | N/A |
| | only) | ANOVA | Low, High, Absence, | |
| | | | Presence) | |
| | | | LFP Frequency (3 levels: | |
| | | | Theta, Alpha, Beta) | |
| 9 | Bradykinesia cohort | Global | Finger (2 levels: | N/A |
| | (averaging off trials) | ANOVA | Index, Middle) | |
| | | | Trial (3 levels: | |
| | | | Trials 1 through 3) | |
| 10 | Bradykinesia cohort (effect | Global | Finger (2 levels: | N/A |
| | of time since stimulation | ANOVA | Index, Middle) | |
| | onset on motor task) | | Epoch (3 levels: | |
| | | | Epochs 1 through 3) | |
| | | | Stimulation (3 levels: | |
| | | | High, Absence, Presence) | |

### 5. EXAMPLES

Clinically effective DBS frequencies provide a very short window (e.g., 5.4 ms at 185 Hz) for acquisition of the biomarker signal due to short inter-pulse intervals and a typical large artifact following each stimulation pulse. As described further herein, one potential solution is to use temporally non-regular patterns of stimulation to provide longer windows to enable sampling of oscillatory activity and DLEPs while maintaining clinical efficacy. By definition, temporally non-regular patterns consist of variable inter-pulse intervals, where longer intervals provide longer windows for biomarker recording. Previous work has demonstrated that the effects of DBS are dependent on the temporal pattern of stimulation; however, embodiments of the present disclosure have demonstrated that altering stimulation patterns provide opportunity to improve both the efficacy (by suppressing oscillatory activity in the basal ganglia) and to enable DLEP quantification, taking advantage of longer periods between pulses. For example, temporally non-regular stimulation patterns can provide greater relief of bradykinesia compared to constant rate DBS, and a computational model predicted that this effect occurred due to greater suppression of beta activity in the basal ganglia. One pattern also had the additional advantage of including 50 ms windows for biomarker quantification. Thus, experiments were conducted to measure the effects of two temporally non-regular stimulation patterns, in persons with PD, on motor symptoms, oscillatory activity in the local field potentials, and DLEPs. These patterns were evaluated for efficacy (degree of symptom relief), efficiency (energy required for delivery of therapy), and feasibility of biomarker quantification (ease of recording) for potential application in closed-loop DBS. Experiments were also conducted to test model predictions from previous work that temporally non-regular patterns of DBS were effective in suppressing beta oscillatory activity in the basal ganglia.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

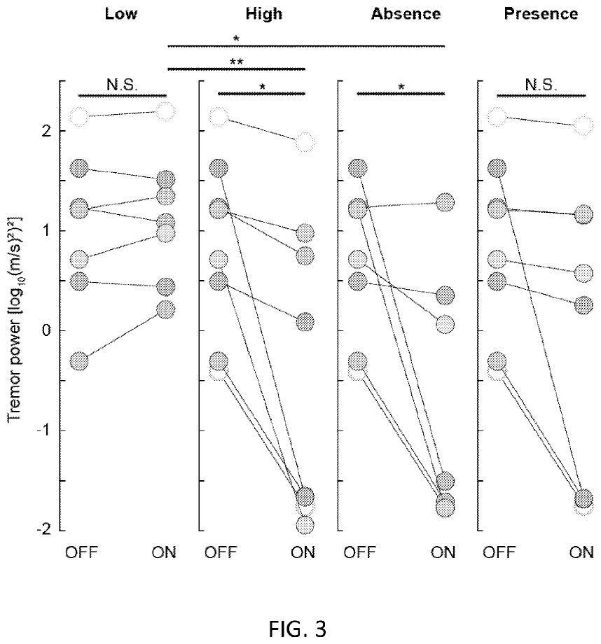

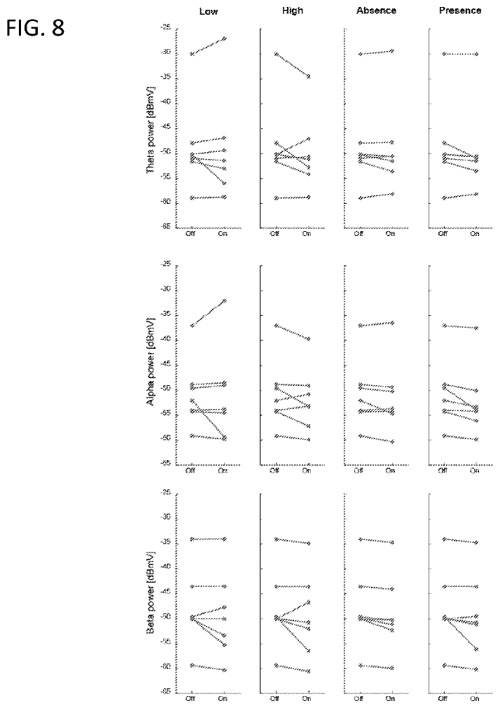

Temporally non-regular DBS relieved tremor without modulation of theta, alpha, and beta power. Experiments were conducted to examine the effect of temporally non-regular patterns of DBS on tremor and LFPs. In the tremor-dominant cohort (FIG. 3), a global 2-way ANOVA detected a significant (F ratio=4.0, p<0.02) interaction term between stimulation pattern and measure (theta power and tremor). Therefore, the data was subdivided between theta power and tremor. A 1-way ANOVA on theta power did not reveal a statistically significant effect of stimulation pattern on theta power (FIG. 8) but there was a significant effect on tremor (F ratio=4.0, p<0.02). Fisher's Protected LSD post-hoc tests distinguished High from Low (p<0.009) and Off (p<0.02), and Absence from Low (p<0.01) and Off (p<0.02). All other pairwise comparisons were not significant (alpha=0.05). The interaction between stimulation pattern and measure in the global ANOVA was thus due to stimulation pattern having an effect on tremor, but not theta power. To investigate further any potential effects of stimulation pattern on the LFP in the tremor-dominant cohort, the analysis was expanded to include alpha and beta power in addition to theta power. A global 2-way ANOVA did not reveal either a significant main effect of stimulation pattern on the LFP nor an interaction between stimulation pattern and LFP frequency (FIG. 8). These results show that DBS reduced tremor in persons with PD without an accompanying modulation in LFP power in the chosen frequency bands, and that this reduction was maintained even when introducing gaps of 50 ms in the stimulation pattern.

### Example 2

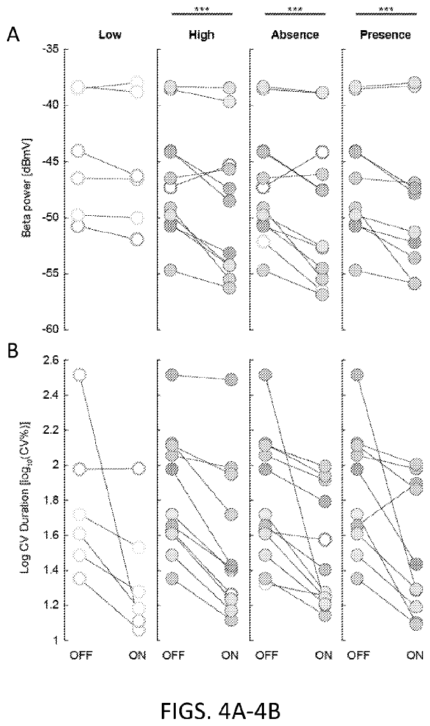

Temporally non-regular DBS relieved bradykinesia and suppressed pathological beta power. Experiments were conducted to examine the effect of temporally non-regular patterns of DBS on bradykinesia and LFPs. In the bradykinesia-dominant cohort (FIG. 4), a global 2-way ANOVA detected a main effect of stimulation pattern across both beta power and bradykinesia (F ratio=11.4, p<6×10⁻⁵). Tukey HSD post-hoc tests distinguished Off from Absence (p<2×10⁻⁴), Presence (p<4×10⁻⁴), and High (p<6×10⁻⁴). All other pairwise comparisons were not significant (alpha=0.05). Since the interaction term between stimulation pattern and measure (beta power and bradykinesia) was not significant, the data was not subdivided further. These results show that DBS reduced bradykinesia and beta power in persons with PD, and that this reduction was maintained even when introducing stimulation gaps of 50 ms.

### Example 3

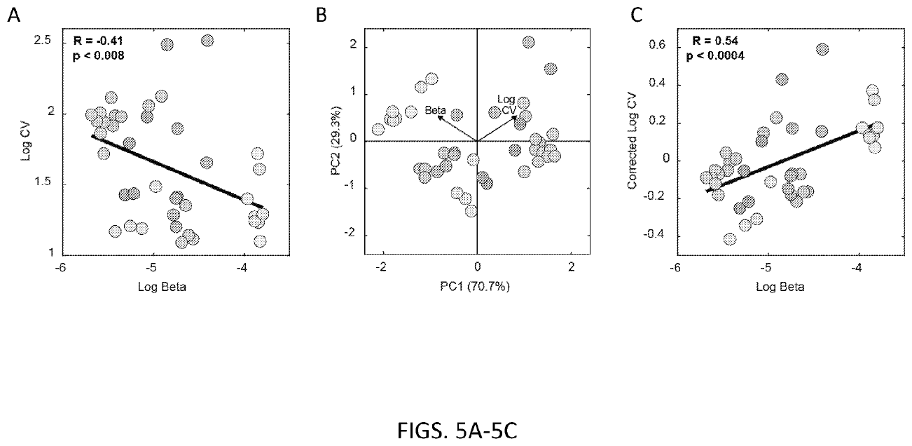

PCA removed interpatient variability and revealed group-level correlations. Experiments were conducted to examine the correlation between beta power and clicking task performance (FIG. 5A) to evaluate the potential of beta power as a biomarker for closed-loop DBS. The initial correlation revealed a trend that contradicted the literature (i.e., a negative correlation). A positive correlation was expected because bradykinesia severity in persons with PD is associated with greater LFP beta power. It was hypothesized that the high interpatient variability was driving this correlation, rather than the pathophysiology of PD. Thus, PCA was used to remove the variability in the clicking task performance that was accounted for by the first principal component, which in this case was the interpatient variability (FIG. 5B). To ensure that each participant contributed equally to the PCA, only participants with Off, High, Absence, and Presence were included (n=10). The PCA-corrected data then revealed the expected positive correlation between beta power and clicking task performance (FIG. 5C): the higher the beta power, the more severe the bradykinesia (R=0.54, p<4×10⁻⁴). These results show that high interpatient variability can drive group-level correlations and obscure underlying pathophysiology. PCA is an effective method for removing interpatient variability when conducting correlations.

### Example 4

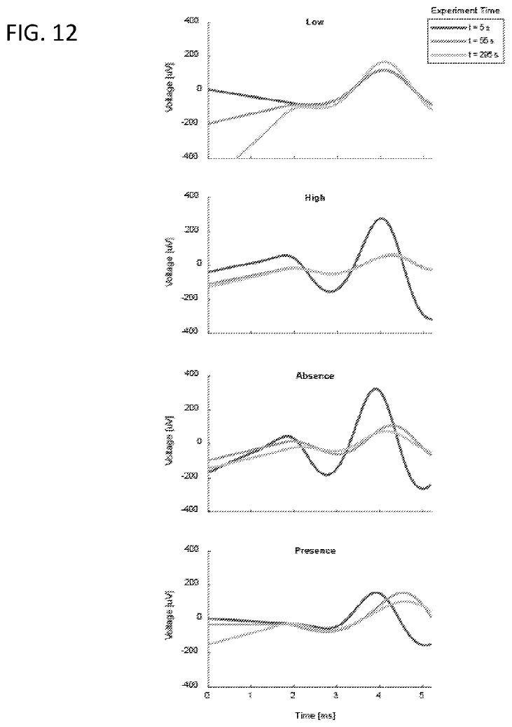

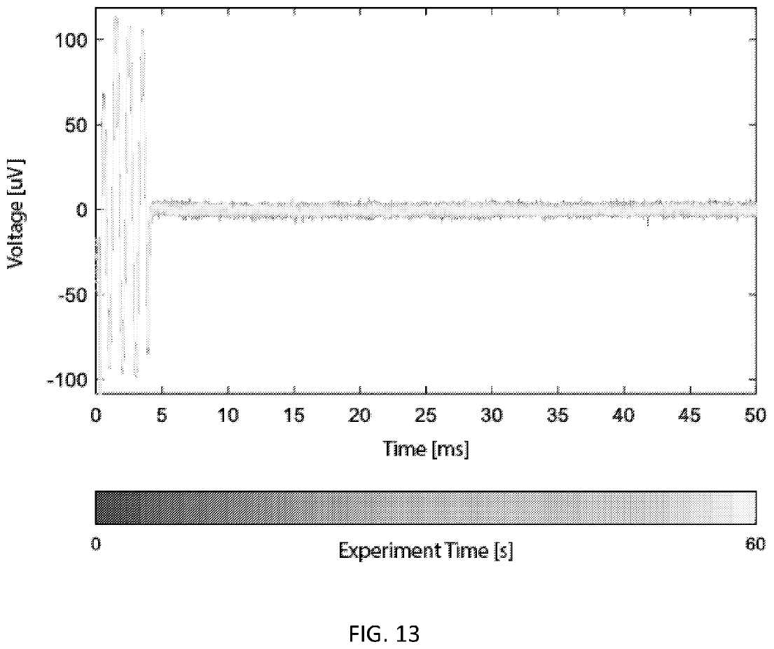

Long-duration DLEPs (up to 50 ms) can be recorded during clinically effective DBS. Experiments were conducted to examine DLEPs during temporally non-regular patterns of DBS. DLEPs were modulated over time during High, Absence, and Presence, but not Low (FIG. 12). The number of DLEP periods and P1 amplitude were examined only during Low and Absence as it was not possible to quantify them during High and Presence due to the short IPIs. Absence produced DLEPs with a greater number of periods compared to Low (FIG. 6A), even though the IPI during the pause in Absence was shorter than the IPI in Low. This was not a ringing artifact from the instrumentation amplifier since it was not present during benchtop bath testing of the instrumentation during Absence stimulation (FIG. 13). The number of DLEP periods present in the first 2 s after stimulation onset during Absence was correlated to the mean SNR of the trial (R=0.75, p<0.002, FIG. 6B). DLEPs recorded during IPG replacements had a lower SNR than those recorded during DBS implantations, suggesting that a deterioration of signal quality over years after lead implantation may result in reduced detection of DLEP periods. Participant 21 (the only participant who enrolled twice) had a much higher SNR during implantation than IPG replacement (approximately 270 and 8, respectively) due to lower signal amplitude and comparable noise. A global 3-way ANOVA detected a significant (F ratio=4.9, p<0.05) interaction term between time, stimulation pattern, and measure (number of periods and P1 amplitude), prompting us to subdivide the data between number of periods (FIG. 6C) and P1 amplitude (FIG. 6D).

A 2-way ANOVA on the number of periods revealed a significant interaction between time and stimulation pattern (F ratio=7.3, p<0.02). Further subdividing the data between stimulation pattern and conducting paired t-tests revealed that the number of DLEP periods decreased between 1 and 51 s for Absence (p<4×10⁻⁴) but not for Low (which did not show any differences between 1 and 51 s). The data was also subdivided between 1 and 51 s. Paired t-tests revealed that stimulation pattern had an effect on the number of DLEP periods at 1 s (p<0.04) but not at 51 s (which did not show any differences between Low and Absence).

A 2-way ANOVA on P1 amplitude revealed a significant interaction between time and stimulation pattern (F ratio=20.6, p<4×10⁻⁴). Further subdividing the data between stimulation pattern and conducting paired t-tests revealed that P1 amplitude decreased between 1 and 51 s for Absence (p<2×10⁻⁴) but not for Low (which did not show any changes between 1 and 51 s). Subdividing the data between 1 and 51 s did not reveal any significant effects of stimulation on P1 amplitude.

P1 latency was analyzed at 1 and 51 s since stimulation onset across all four stimulation patterns (FIG. 6E). A global 2-way ANOVA revealed a significant interaction between time and stimulation pattern (F ratio=6.0, p<0.003). Therefore, the data was subdivided by stimulation pattern. For each stimulation pattern, a paired t-test was conducted comparing P1 latency at 1 and 51 s since stimulation onset. P1 latency increased between 1 and 51 s for High (p<4×10⁻⁴), Absence (p<0.002), and Presence (p<2×10⁻⁴), but not for Low (which did not show any differences between 1 and 51 s). The data was also subdivided by time. A 1-way ANOVA detected an effect of stimulation (F ratio=4.4, p<0.02) on P1 latency at 1 s. Tukey HSD post-hoc tests distinguished Low from High (p<0.02), Absence (p<0.05), and Presence (p<0.03). At 51 s, there was no statistically significant effect of stimulation on P1 latency (differences between stimulation patterns at 51 s seemed to mirror those at 1 s, albeit less marked).

These results show that it is feasible to monitor the entirety of DLEPs during clinically effective DBS by introducing stimulation gaps of 50 ms. DLEPs recorded during Absence showed the same modulation as those recorded during High (smaller amplitude and longer latency). Absence revealed the number of DLEP periods as an additional characteristic that is modulated during clinically effective DBS.

It is understood that the foregoing detailed description and accompanying examples are merely illustrative and are not to be taken as limitations upon the scope of the disclosure, which is defined solely by the appended claims and their equivalents. Various changes and modifications to the disclosed embodiments will be apparent to those skilled in the art.

1. A method of administering brain stimulation therapy to a subject, the method comprising:
delivering a temporally non-regular pattern of electrical stimulation to the subject's brain;
measuring at least one neural biomarker in the local field potential (LFP); and
treating at least one symptom in the subject.

2. The method of claim 1, wherein administering the brain stimulation to the subject comprises administering deep brain stimulation (DBS), epidural cortical stimulation, epicortical cortical stimulation, transcranial electrical stimulation, and/or transcranial magnetic stimulation.

3. The method of claim 1, wherein administering the brain stimulation to the subject comprises administering closed-loop brain stimulation.

4. The method of claim 1, wherein the at least one neural biomarker comprises deep brain stimulation local evoked potential (DLEP) and/or oscillatory activity.

5. The method of claim 1, wherein measuring the at least one neural biomarker comprises recording single-neuron activity.

6. The method of claim 1, wherein the subject has or is suspected of having a neurological disease or disorder.

7. (canceled)

8. The method of claim 1, wherein the temporally non-regular pattern of electrical stimulation comprises at least one time period in which electrical stimulation is absent.

9. The method of claim 8, wherein the at least one time period in which electrical stimulation is absent is from about 20 ms to about 200 ms in length.

10. The method of claim 8, wherein the at least one time period in which electrical stimulation is absent occurs about every 100 ms to about every 5000 ms.

11. The method of claim 1, wherein the at least one neural biomarker comprises the DLEP, and wherein the DLEP is measured during the at least one time period in which electrical stimulation is absent.

12. The method of claim 1, wherein the at least one neural biomarker comprises oscillatory activity, and wherein oscillatory activity is measured during the at least one time period in which electrical stimulation is absent.

13. (canceled)

14. The method of claim 1, wherein treating at least one symptom in the subject comprises suppressing one or more of bradykinesia, tremor, and/or rigidity.

15. The method of claim 1, wherein treating at least one symptom in the subject comprises suppressing at least one of alpha, beta, gamma, and/or theta oscillations.

16. (canceled)

17. The method of claim 1, wherein the method further comprises adjusting one or more stimulation parameters based on information obtained from measuring at least one neural biomarker.

18-19. (canceled)

20. The method of claim 1, wherein the at least one neural biomarker comprises DLEP and/or oscillatory activity, and wherein the information obtained from measuring the DLEP and/or oscillatory activity indicates where to place an initial electrode in a subject's brain.

21. The method of claim 1, wherein the at least one neural biomarker comprises DLEP and/or oscillatory activity, and wherein the information obtained from measuring the DLEP and/or oscillatory activity facilitates optimizing electrode configuration in a subject's brain.

22. (canceled)

23. A system for administering brain stimulation therapy to a subject, the system comprising:
at least one implantable electrode configured to deliver electrical stimulation and to record electrical signals in the subject's brain; and
a pulse generator electronically coupled to the at last one implantable electrode, wherein the pulse generator is configured to deliver a temporally non-regular pattern of electrical stimulation and measure at least one neural biomarker in the local field potential (LFP).

24. The system of claim 23, wherein the system administers at least one of deep brain stimulation (DBS), epidural cortical stimulation, epicortical cortical stimulation, transcranial electrical stimulation, and/or transcranial magnetic stimulation to the subject.

25. (canceled)

26. The system of claim 23, wherein the at least one neural biomarker comprises deep brain stimulation local evoked potential (DLEP) and/or oscillatory activity.

27. (canceled)

28. The system of claim 23, wherein the temporally non-regular pattern of electrical stimulation comprises at least one time period in which electrical stimulation is absent.

29-30. (canceled)
